SEQUENCE LISTING (13 sequences)

SEQ ID NO: 1 - protein, 263 aa, Homo sapiens
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 2 - protein, 218 aa, Homo sapiens
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFR

SEQ ID NO: 3 - protein, 6 aa, Homo sapiens
GRGDSP

SEQ ID NO: 4 - protein, 7 aa, Homo sapiens
GNGRAHA

SEQ ID NO: 5 - protein, 10 aa, Homo sapiens
GALNGRSHAG

SEQ ID NO: 6 - protein, 7 aa, Homo sapiens
GCNGRCG

SEQ ID NO: 7 - protein, 14 aa, Homo sapiens
GCNGRCVSGCAGRC

SEQ ID NO: 8 - protein, 10 aa, Homo sapiens
GCVLNGRMEC

SEQ ID NO: 9 - protein, 221 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRRGD

SEQ ID NO: 10 - DNA, 33 bp, synthetic construct
  source 1..33: mol_type = other DNA; note = tTF Primer 1
catgccatgggatcaggcactacaaatactgtg

SEQ ID NO: 11 - DNA, 29 bp, synthetic construct
  source 1..29: mol_type = other DNA; note = Primer 2
cgggatcctattatctgaattcccctttc

SEQ ID NO: 12 - DNA, 33 bp, synthetic construct; identical to SEQ ID NO: 10
  source 1..33: mol_type = other DNA; note = tTF-RGD Primer 1

SEQ ID NO: 13 - DNA, 43 bp, synthetic construct
  source 1..43: mol_type = other DNA; note = Primer 3
cgggatcctattatggagaatcacctcttcctctgaattcccc

(19) United States
(12) Patent Application Publication
(10) Pub. No.: US 2026/0226138 A1
(43) Pub. Date: Aug. 6, 2026

(54) COMBINATION TUMOR THERAPY WITH THROMBOSIS INITIATION AND PLATELET RECRUITMENT

(71) Applicant: Wisconsin Alumni Research Foundation, Madison, WI (US)

(72) Inventors: Quanyin Hu, Madison, WI (US); Yixin Wang, Madison, WI (US)

(73) Assignee: Wisconsin Alumni Research Foundation, Madison, WI (US)

(21) Appl. No.: 19/127,975

(22) PCT Filed: Nov. 8, 2023
(86) PCT No.: PCT/US2023/079086

§ 371 Date: May 7, 2025

Related U.S. Application Data
(60) Provisional application No. 63/423,884, filed on Nov. 9, 2022.

(51) Int. Cl. C07K 14/745; A61K 31/337; A61K 38/00; A61K 39/00; A61K 47/68; A61K 47/69; A61P 35/00; C07K 16/28

(52) U.S. Cl. CPC C07K 14/745; A61K 31/337; A61K 47/6843; A61K 47/6901; A61P 35/00; C07K 16/2818; A61K 38/00; A61K 2039/505; C07K 2317/76; C07K 2319/33

(57) ABSTRACT

Described herein is a method of treating a human patient with a solid tumor, the method including administering to the human patient an effective amount of a fusion polypeptide comprising a tissue factor fragment and a covalently linked tumor vessel endothelial cell binding peptide, wherein the effective amount activates coagulation at the solid tumor site; waiting for a period of time; and administering to the human patient an effective amount of a composition comprising a platelet cell or a platelet membrane. The platelet cell includes a covalently linked immune checkpoint inhibitor, and the platelet membrane coats a chemotherapeutic agent nanoparticle.

### CROSS-REFERENCE TO RELATED APPLICATIONS

This application claims priority to U.S. Provisional Application 63/423,884 filed on Nov. 9, 2022, which is incorporated herein by reference in its entirety.

### SEQUENCE LISTING

The Instant Application contains a Sequence Listing which has been submitted electronically in XML format and is hereby incorporated by reference in its entirety. Said XML copy, created on Oct. 19, 2023, is named “SEQ_LIST-107668_176” and is 13.4 KB (13,740 bytes) in size. The Sequence Listing does not go beyond the disclosure in the application as filed.

### FIELD OF THE DISCLOSURE

The present disclosure is related to a therapeutic regimen that combines initiation of thrombosis with administration of an engineered platelet or platelet-derived therapeutic.

### BACKGROUND

Immune checkpoint blockade (ICB)-based immunotherapy has revolutionized the paradigm of tumor therapy. Blocking the interaction between checkpoint receptors on immune cells and their ligands on tumor cells with immune checkpoint inhibitors (ICIs) contributes to the restoration of T cell function for eradicating tumors. However, the clinical application of ICIs has been associated with various immune-related adverse events (iRAEs). Moreover, although encouraging therapeutic outcomes have been achieved from the combination ICB therapy with anti-PD-L1/PD-1 and anti-CTLA-4 monoclonal antibodies, it is also reported to induce more toxicity. Antibodies administered via conventional systemic routes do not preferentially accumulate in the tumor area, resulting in the off-target binding to normal tissues, which may cause severe iRAEs and compromise the therapeutic efficacy. Also, previous studies have validated that the therapeutic efficacy of ICIs largely depending on how they are delivered, and locoregional administration such as intratumoral injection contributed to longer tumor retention and robust systemic anti-tumoral immunity compared to intraperitoneal injection. Moreover, the reduction in the required dose by intratumoral injection will minimize the potential off-target toxicities on normal tissues.

Even though intratumoral injection is a direct route that guarantees the access of drugs to tumor sites and is widely adopted in animal-based studies, the difficulties in the implementation of intratumoral injection to deep-seated tumors or tumors with small volumes restrict its application in the clinic. Besides, this administration strategy relies on the skills of professionals and the guidance of imaging techniques. Active targeting delivery systems, most of which are designed based on ligand-receptor interaction, in which specific ligands are decorated on the delivery systems to bind to the disease-overexpressing receptors, can achieve local accumulation and site-specific ICI delivery after being administered systemically. However, many receptors are not tumor-specific and are also present on normal tissues, which may lead to off-target effects and systemic toxicity. For other delivery systems that target unique receptors with high tumor specificity, the significant tumor heterogeneity remains an intractable problem that may lead to the failure of efficient targeting as expected. Furthermore, the conventional active targeting strategy does not allow the subsequent accumulation of delivery systems after the saturation of target receptor binding, which may dampen the efficiency of ligand-receptor-oriented delivery systems even with repetitive dosing. Therefore, there is a clear and urgent need for the development of novel platforms that can overcome the limitations of current active targeting strategies for ICI delivery.

### BRIEF SUMMARY

In an aspect, a method of treating a human patient with a solid tumor comprises administering to the human patient an effective amount of a fusion polypeptide comprising a tissue factor fragment and a covalently linked tumor vessel endothelial cell binding peptide, wherein the effective amount activates coagulation at the solid tumor site; waiting for a period of time; and administering to the human patient an effective amount of a composition comprising a platelet cell or a platelet membrane, wherein the platelet cell comprises a covalently linked immune checkpoint inhibitor, and wherein the platelet membrane coats a chemotherapeutic agent nanoparticle.

### BRIEF DESCRIPTION OF THE DRAWINGS

FIGS. 1A-E show the characterization of the thrombosis-mediated navigation system. FIG. 1A is a schematic of the thrombosis-mediated navigation system for platelets. tTF-RGD administered either by intravenous (i.v.) or peritumoral (p.t.) injection targets tumor endothelial cells and initiates coagulation cascade to recruit platelets. Platelets decorated with aPD-1 antibody (P-aPD-1) are activated during the coagulation process to release aPD-1 in the form of PMP-aPD-1. FIG. 1B shows an SDS-PAGE analysis of tTF and tTF-RGD. FIG. 1C shows the ability of tTF and tTF-RGD to promote the proteolytic activation of FX by FVIIa. Data are mean±s.d. n=3 per group. FIG. 1D shows ex vivo photographs of tumor tissues at 24 h post intratumoral injection of BSA, tTF and tTF-RGD. FIG. 1E shows hematoxylin and eosin (H&E) staining of tumor tissues at 24 after three doses of i.v. injection of proteins.

FIGS. 2A-H show the characterization of tumor targeting and coagulation initiation capabilities of tTF-RGD. FIG. 2A shows fluorescence imaging of mice at different time points after i.v. injection of Cy5.5-labeled tTF or tTF-RGD (n=3 per group). tTF-RGD=1.25 mg/kg. FIG. 2B shows ex vivo fluorescence imaging of tumor tissues at 24 h. FIG. 2C shows the mean fluorescence intensities of tumors shown in 2B. *P<0.05, unpaired Student's t-test. FIG. 2D shows a schematic illustration of the experimental design to examine the coagulation initiation capabilities of tTF-RGD. FIG. 2E illustrates the underlying mechanism of tTF-RGD to trigger activation of coagulation process and attract fibrinogen. FIG. 2F shows representative fluorescence images showing the biodistribution of Cy5.5-labeled fibrinogen at 24 h post i.v. injection (n=3 per group). tTF-RGD=1.25 mg/kg. FIG. 2G shows ex vivo fluorescence imaging of tumor tissues at 24 h. FIG. 2H shows the mean fluorescence intensities (MFI) of tumors shown in 2G. Data are presented as mean±SEM. **P<0.01, one-way ANOVA followed by multiple comparison test.

FIGS. 3A-J show the characterization of platelet recruitment capabilities of tTF-RGD. FIG. 3A is a schematic illustration of the experimental design to examine the recruitment of platelets to tumor areas after p.t. injection of tTF-RGD. FIG. 3B shows fluorescence imaging of mice at different time points after injection of Cy5.5-labeled platelets (n=3 per group). tTF-RGD=0.4 mg/kg. FIG. 3C shows ex vivo fluorescence imaging of tumor tissues at 24 h. FIG. 3D shows the mean fluorescence intensities of tumors shown in 3C. *P<0.05, one-way ANOVA followed by multiple comparison test. FIG. 3E shows a schematic illustration of the experimental design to examine the recruitment of platelets to tumor areas after i.v. injection of tTF-RGD. FIG. 3F shows fluorescence imaging of mice at different time points after injection of Cy5.5-labeled platelets (n=3 per group). tTF-RGD=1.25 mg/kg. FIG. 3G shows ex vivo fluorescence imaging of tumor tissues at 24 h. FIG. 3H shows the mean fluorescence intensities of tumor shown in 3G. FIGS. 3I and 3J show accumulation of platelets in tumors resected from mice treated with p.t. injected tTF-RGD+i.v. injected platelets (31) or i.v. injected tTF-RGD+i.v. injected platelets (3J). Data are presented as mean±SEM. *P<0.05, one-way ANOVA followed by multiple comparison test.

FIGS. 4A-F show an evaluation of therapeutic efficacy of p.t. injected tTF-RGD+i.v. injected P-aPD-1 in CT26 subcutaneous tumor model. FIG. 4A is a schematic illustration of the timeline of model establishment and treatment strategy. FIG. 4B shows tumor growth of individual mice in different groups (aPD-1=1 mg/kg, tTF or tTF-RGD=0.4 mg/kg). Cure rate (CR) in tTF-RGD+P-aPD-1 treatment group is 3/9. n=6-9. FIG. 4C shows tumor volume changes after different treatment. Data are presented as mean±SEM. **P<0.01, One-way ANOVA followed by multiple comparison test. FIG. 4D shows survival time of mice receiving different treatments. **P<0.01. Data were analyzed with Log-rank test. FIG. 4E shows bioluminescence images of native mice or tumor-free mice re-challenged with 106 of CT26-luc cells (n=3). FIG. 4F shows quantitative region-of-interest analysis of bioluminescence intensities in 4E. Data are presented as mean±SEM. ***P<0.001, unpaired Student's t-test.

FIGS. 5A-D show an evaluation of therapeutic efficacy of i.v. injected tTF-RGD+i.v. injected P-aPD-1 in CT26 subcutaneous tumor model. FIG. 5A is a schematic illustration of the timeline of model establishment and treatment strategy. FIG. 5B shows tumor growth of individual mice in different groups (aPD-1=0.3 mg/kg, tTF or tTF-RGD=1.25 mg/kg). n=6-8. FIG. 5C shows tumor volume changes after different treatment. Data are presented as mean±SEM. **P<0.01. One-way ANOVA followed by multiple comparison test. FIG. 5D shows survival time of mice receiving different treatments. Data were analyzed with Log-rank test. **P<0.01.

FIGS. 6A-I show the evaluation of the immunocellular composition and cytokine secretion after different treatments. FIG. 6A shows representative flow cytometry plots of CD8⁺ T cells (gated on CD3⁺ T cells) in tumor tissues after treatment of p.t. injected tTF-RGD+i.v. injected P-aPD-1 (n=4). FIG. 6B shows quantitative analysis of the percentage of CD3⁺CD8⁺ T cells. FIG. 6C shows the percentage of IFN-γ+ populations in CD3⁺CD8⁺ T cells. *P<0.05. ***P<0.001, one-way ANOVA followed by multiple comparison test. FIG. 6D shows quantification of cytokines in tumor supernatant (n=4).

FIG. 6E shows representative flow cytometry plots of CD8⁺ T cells (gated on CD3⁺ T cells) in tumor tissues after treatment of i.v. injected tTF-RGD+i.v. injected P-aPD-1. FIG. 6F shows a quantitative analysis of the percentage of CD3⁺CD8⁺ T cells. FIG. 6G shows the percentage of IFN-γ+ populations in CD3⁺CD8⁺ T cells. FIGS. 6H and 6I show the levels of IFN-γ (FIG. 6H) and TNF-α (FIG. 6I) in tumor supernatant detected by enzyme-linked immunosorbent assay (ELISA) (n=4). Data are presented as mean±SEM. **P<0.01. ***P<0.001, one-way ANOVA followed by multiple comparison test.

FIGS. 7A-H shows an evaluation of therapeutic efficacy of paclitaxel (PTX)-loaded platelet membrane (PLM)-coated nanoparticles (NPs) enhanced by tTF-RGD. FIG. 7A is a schematic illustration of PM-NP. PM (platelet membrane). FIG. 7B shows TEM images of NPs, PM and PM-NPs. FIG. 7C shows the establishment of PDX model and the treatment strategy. FIG. 7D shows H&E staining of tumor tissues after receiving tTF-RGD+PM-NP. FIG. 7E shows fluorescence imaging of mice at 24 h after i.v. injection of DiR-loaded PM-NP and the ex vivo images of tumors (n=3). FIG. 7F shows the mean fluorescence intensities of tumor shown in (E). **P<0.01, one-way ANOVA followed by multiple comparison test. FIG. 7G shows photographs of tumors on Day 38. Scale bar=1 cm. FIG. 7H shows tumor growth curve of mice received three doses of different treatment. N=7 in all groups. Data are presented as mean±SEM. **P<0.01, one-way ANOVA followed by multiple comparison test.

The above-described and other features will be appreciated and understood by those skilled in the art from the following detailed description, drawings, and appended claims.

### DETAILED DESCRIPTION

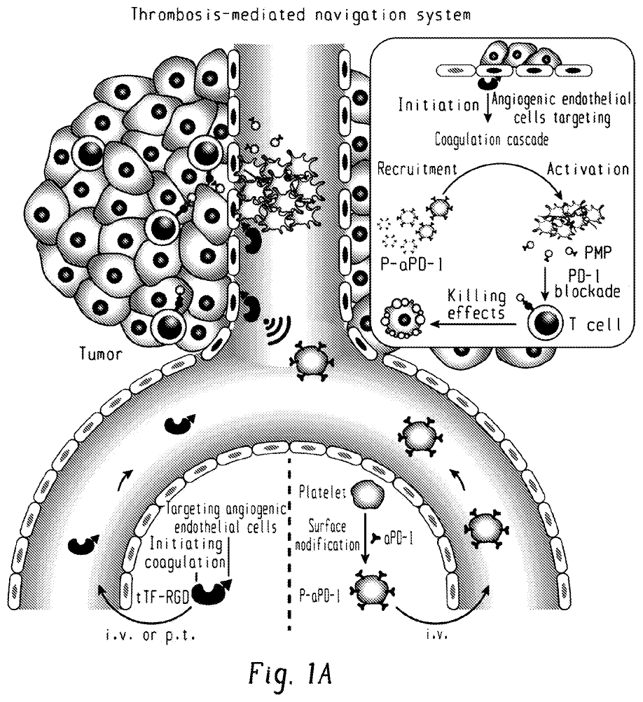

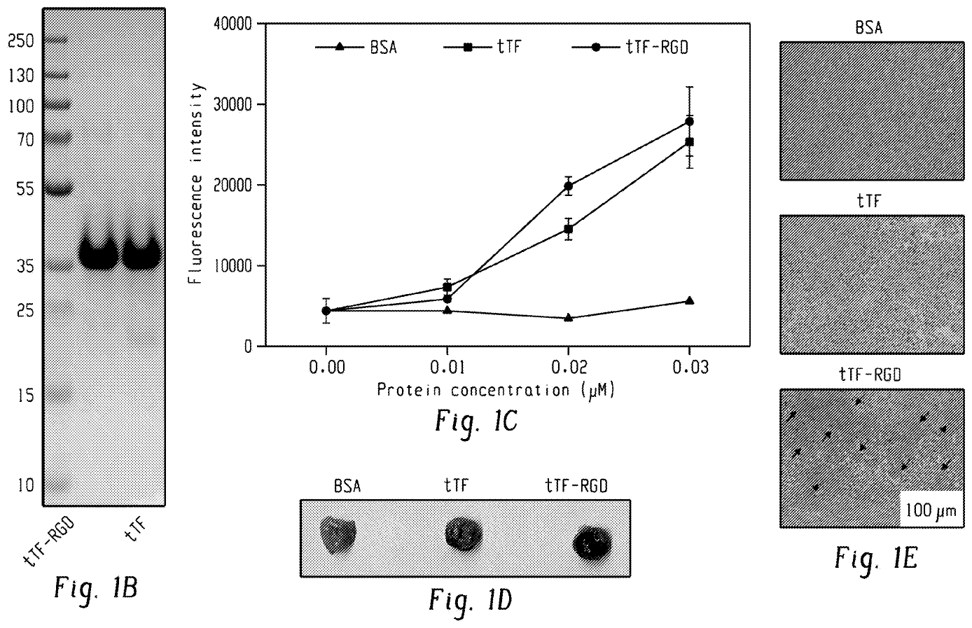

Specifically described herein is the creation of a “cellular hive” that can navigate anti-PD-1 antibody-engineered platelets (designated P-aPD-1) or platelet membrane coated agents homing to a tumor site and unload the therapeutic cargoes locally. Tissue factor, as one initiator of blood coagulation, can activate the extrinsic coagulation pathway and induce the transformation of prothrombin to thrombin, in which process substantial amounts of platelets are recruited to form a thrombus. To mimic the inherent coagulation process, a fused protein truncated tissue factor-Arg-Gly-Asp (RGD) (tTF-RGD)—was synthesized, which is capable of initiating the coagulation cascade upon the interaction between the RGD motif and αᵥβ₃ integrin on endothelial cells of tumor neovasculature. RGD is an extensively investigated motif that can bind to integrin αvβ3 overexpressed on tumor cells and endothelial cells of tumor neovasculature. In this design, RGD was leveraged to shuttle tTF to the angiogenic tumor blood vessel to initiate the coagulation signaling cascade. Without being held to theory, it is believed that this strategy will bypass the tumor heterogenicity and binding saturation since it does not require substantial accumulation of tTF-RGD to trigger thrombus formation. The tTF-RGD can be injected either by peritumoral or intravenous injection to trigger the coagulation cascade only in the tumor region, amplifying the signals to recruit platelets to form a thrombus (FIG. 1A). P-aPD-1 that are injected subsequently can respond to this physiological signaling, actively participating in the coagulation cascade, and enriching at the tumor site. The coagulation cascade further triggers the activation of platelet to secrete platelet-derived microparticles (PMPs) and releases aPD-1 antibodies within the tumor site to reinvigorate T cells for enhanced immune response. To further extend the treatment strategy to other therapeutic modalities, the chemotherapeutic paclitaxel (PTX) was encapsulated in dextran nanoparticles, and the nanoparticles were coated with platelet membrane (PM) to form PM-NP/PTX. PM-NPs hold the potential to migrate in response to platelet-attracting signals such as tTF-RGD-mediated coagulation. The enhanced tumor accumulation and antitumoral efficacy were demonstrated on a patient-derived xenograft (PDX) breast cancer model which substantiated the clinical treatment potential of the treatment approach. Thus, described herein is a strategy to enhance tumor accumulation of different therapeutics which can be easily adapted to other treatment modalities. Furthermore, this treatment approach can mimic the physiological signaling cascade to artificially create a tumor-targeting environment, which can enhance the tumor-selective accumulation of therapeutics and bypass the limitations of conventional active targeting strategy.

In an aspect, a method of treating a human patient with a solid tumor comprises administering to the human patient an effective amount of a fusion polypeptide comprising a tissue factor fragment and a covalently linked tumor vessel endothelial cell binding peptide, wherein the effective amount activates coagulation at the solid tumor site; waiting for a period of time; and administering to the human patient an effective amount of a composition comprising a platelet cell or a platelet membrane. The platelet cell comprises a covalently linked immune checkpoint inhibitor, and the platelet membrane coats a chemotherapeutic agent nanoparticle.

Described herein is thus a treatment regimen for the treatment of solid tumors. A first component of the treatment regimen is a fusion polypeptide comprising a tissue factor fragment and a covalently linked tumor vessel endothelial cell binding peptide, linked directly or via a 1-15 amino acid linker. Such peptides are described in U.S. Pat. No. 7,618,943, incorporated herein by reference for its disclosure of fusion polypeptides such as tTF-NGR.

Tissue factor (SEQ ID NO: 1) is a peptide that activates blood clotting. In an aspect, a tissue factor fragment (tTF) comprises tTF1-218, (SEQ ID NO: 2) which is the 218 N-terminal amino acids of TF corresponding to the extracellular domain. A tissue factor fragment can also include fragments can be used that lack up to 8 amino acids at the C-terminus (tTF₁₋₂₁₀), such as (tTF₁₋₂₁₄). In an aspect, tissue factor fragment comprises amino acids 1-210 to 1-218 of SEQ ID NO: 1, specifically tTF₁₋₂₁₈, (SEQ ID NO: 2). Amino acid sequences with 90, 95 or 98% sequence identity to SEQ ID Nos. 1 and 2 may also be employed, so long as they function to activate blood clotting.

(SEQ ID NO: 1)
Ser Gly Thr Thr Asn Thr Val Ala Ala Tyr Asn Leu Thr Trp Lys Ser
Thr Asn Phe Lys Thr Ile Leu Glu Trp Glu Pro Lys Pro Val Asn Gln
Val Tyr Thr Val Gln Ile Ser Thr Lys Ser Gly Asp Trp Lys Ser Lys
Cys Phe Tyr Thr Thr Asp Thr Glu Cys Asp Leu Thr Asp Glu Ile Val
Lys Asp Val Lys Gln Thr Tyr Leu Ala Arg Val Phe Ser Tyr Pro Ala
Gly Asn Val Glu Ser Thr Gly Ser Ala Gly Glu Pro Leu Tyr Glu Asn
Ser Pro Glu Phe Thr Pro Tyr Leu Glu Thr Asn Leu Gly Gln Pro Thr
Ile Gln Ser Phe Glu Gln Val Gly Thr Lys Val Asn Val Thr Val Glu
Asp Glu Arg Thr Leu Val Arg Arg Asn Asn Thr Phe Leu Ser Leu Arg
Asp Val Phe Gly Lys Asp Leu Ile Tyr Thr Leu Tyr Tyr Trp Lys Ser
Ser Ser Ser Gly Lys Lys Thr Ala Lys Thr Asn Thr Asn Glu Phe Leu
Ile Asp Val Asp Lys Gly Glu Asn Tyr Cys Phe Ser Val Gln Ala Val
Ile Pro Ser Arg Thr Val Asn Arg Lys Ser Thr Asp Ser Pro Val Glu
Cys Met Gly Gln Glu Lys Gly Glu Phe Arg Glu Ile Phe Tyr Ile Ile
Gly Ala Val Val Phe Val Val Ile Ile Leu Val Ile Ile Leu Ala Ile
Ser Leu His Lys Cys Arg Lys Ala Gly Val Gly Gln Ser Trp Lys Glu
Asn Ser Pro Leu Asn Val Ser
(SEQ ID NO: 2)
Ser Gly Thr Thr Asn Thr Val Ala Ala Tyr Asn Leu Thr Trp Lys Ser
Thr Asn Phe Lys Thr Ile Leu Glu Trp Glu Pro Lys Pro Val Asn Gln
Val Tyr Thr Val Gln Ile Ser Thr Lys Ser Gly Asp Trp Lys Ser Lys
Cys Phe Tyr Thr Thr Asp Thr Glu Cys Asp Leu Thr Asp Glu Ile Val
Lys Asp Val Lys Gln Thr Tyr Leu Ala Arg Val Phe Ser Tyr Pro Ala
Gly Asn Val Glu Ser Thr Gly Ser Ala Gly Glu Pro Leu Tyr Glu Asn
Ser Pro Glu Phe Thr Pro Tyr Leu Glu Thr Asn Leu Gly Gln Pro Thr
Ile Gln Ser Phe Glu Gln Val Gly Thr Lys Val Asn Val Thr Val Glu
Asp Glu Arg Thr Leu Val Arg Arg Asn Asn Thr Phe Leu Ser Leu Arg
Asp Val Phe Gly Lys Asp Leu Ile Tyr Thr Leu Tyr Tyr Trp Lys Ser
Ser Ser Ser Gly Lys Lys Thr Ala Lys Thr Asn Thr Asn Glu Phe Leu
Ile Asp Val Asp Lys Gly Glu Asn Tyr Cys Phe Ser Val Gln Ala Val
Ile Pro Ser Arg Thr Val Asn Arg Lys Ser Thr Asp Ser Pro Val Glu
Cys Met Gly Gln Glu Lys Gly Glu Phe Arg

The tumor vessel endothelial cell binding peptide has a length of 3-30 amino acids and binds tumor vessel endothelial cells with high specificity. Such peptides can be isolated from peptide libraries by methods that are usual in the state of the art. They can have a linear or cyclic structure. Exemplary tumor vessel endothelial cell binding peptides include the amino acid sequence RGD or NGR. Both sequences specifically bind to integrins, especially αᵥβ₃ and αᵥβ₅ integrins (RGD peptides), and as cell adhesion motifs (NGR peptides). Exemplary tumor vessel endothelial cell binding peptides include GRGDSP (SEQ ID NO: 3), GNGRAHA (SEQ ID NO:4) and GALNGRSHAG (SEQ ID NO:5) and the cyclic peptides with the sequences GCNGRCG (SEQ ID NO:6), GCNGRCVSGCAGRC (SEQ ID NO: 7) and GCVLNGRMEC (SEQ ID NO:8).

In an aspect, the peptide comprises (SEQ ID NO: 9).

(SEQ ID NO: 9)
Ser Gly Thr Thr Asn Thr Val Ala Ala Tyr Asn Leu Thr Trp Lys Ser
Thr Asn Phe Lys Thr Ile Leu Glu Trp Glu Pro Lys Pro Val Asn Gln
Val Tyr Thr Val Gln Ile Ser Thr Lys Ser Gly Asp Trp Lys Ser Lys
Cys Phe Tyr Thr Thr Asp Thr Glu Cys Asp Leu Thr Asp Glu Ile Val
Lys Asp Val Lys Gln Thr Tyr Leu Ala Arg Val Phe Ser Tyr Pro Ala
Gly Asn Val Glu Ser Thr Gly Ser Ala Gly Glu Pro Leu Tyr Glu Asn
Ser Pro Glu Phe Thr Pro Tyr Leu Glu Thr Asn Leu Gly Gln Pro Thr
Ile Gln Ser Phe Glu Gln Val Gly Thr Lys Val Asn Val Thr Val Glu
Asp Glu Arg Thr Leu Val Arg Arg Asn Asn Thr Phe Leu Ser Leu Arg
Asp Val Phe Gly Lys Asp Leu Ile Tyr Thr Leu Tyr Tyr Trp Lys Ser
Ser Ser Ser Gly Lys Lys Thr Ala Lys Thr Asn Thr Asn Glu Phe Leu
Ile Asp Val Asp Lys Gly Glu Asn Tyr Cys Phe Ser Val Gln Ala Val
Ile Pro Ser Arg Thr Val Asn Arg Lys Ser Thr Asp Ser Pro Val Glu
Cys Met Gly Gln Glu Lys Gly Glu Phe Arg Arg Gly Asp

The fusion polypeptide can be produced using recombinant production in cells such as E. coli, yeast cells, and animal cell lines, such as CHO- or COS-cells. The fusion polypeptides can also be produced by a chemical coupling of individual peptides. Thus, individual peptides can be produced by methods that are conventional in the state of the art, e.g., by chemical synthesis or by heterologous expression, and are then joined together by coupling.

The fusion polypeptide may be in the form of a pharmaceutical composition comprising, for example, a pharmaceutically acceptable carrier, diluent and/or adjuvant.

A second component of the treatment regimen comprises a platelet cell or a platelet membrane. In an aspect, the second component is a platelet cell comprising a covalently linked ICI. The platelet cell and the ICI can be covalently linked via a chemical linker moiety.

In one embodiment, the platelet cell is a human platelet cell. In one embodiment, the platelet cell is an autologous platelet cell. Platelet cells can be isolated from whole blood by centrifuging whole blood and separating the platelet-rich plasma. The platelets can then be separated from the plasma by centrifugation.

Immune checkpoints refer to a plurality of inhibitory pathways hardwired into the immune system that are crucial for maintaining self-tolerance and modulating the duration and amplitude of physiological immune responses in peripheral tissues in order to minimize collateral tissue damage. Tumors co-opt certain immune-checkpoint pathways as a major mechanism of immune resistance, particularly against T cells that are specific for tumor antigens. Because many of the immune checkpoints are initiated by ligand-receptor interactions, they can be readily blocked by antibodies or modulated by recombinant forms of ligands or receptors. In an embodiment, the ICI is an antibody that specifically binds CD25, PD-1, PD-L1, PD-L2, CTLA-4, immunoglobulin receptor (KIR), LAG-3, TIM-3, 4-1BB, 4-1BBL, GITR, CD40, CD40L, OX40, OX40L, CXCR2, B7-H3, B7-H4, BTLA, HVEM, CD28, A2aR, CD27, CD70, TCR ICOS, CD80, CD86, ICOS-L, CD70, Gal-9, VISTA, CD-137, CD155, CD266, PVR, PVR-2, CD47, CD160, NT5E, CD96, TNFRSF18, or a combination comprising one or more of the foregoing. In an embodiment, the ICI antibody is a whole antibody, an antibody fragment, or a peptide.

Exemplary immune checkpoint inhibitors include cemiplimab-rwle, nivolumab, pembrolizumab, pidilizumab, MEDI-0680, PDR001, REGN2810, and BGB-108, AMP-224, an immunoadhesin, BMS-936559, atezolizumab, YW243.55.S70, MDX-1105, MEDI4736, durvalumab, avelumab, ipilimumab, tremelimumab, BMS-986016, urelumab, TRX518, dacetuzumab, lucatumumab, SEA-CD40, CP-870,893, MED16469, MOXR0916, MSB001078C, or a combination comprising one or more of the foregoing.

In an embodiment, the ICI is a PD-1 binding molecule (e.g., antagonist), and in particular, is an anti-PD-1 antibody (e.g., a human antibody, a humanized antibody, or a chimeric antibody). Exemplary anti-PD-1 antibodies include REGN2810 (cemiplimab), MDX-1106 (nivolumab), MK-3475 (pembrolizumab), CT-011 (pidilizumab), MEDI-0680 (AMP-514), PDR001, and BGB-108 (Tislelizumab). In an embodiment, the PD-1 binding molecule is an immunoadhesin (e.g., an immunoadhesin comprising an extracellular or PD-1 binding portion of PD-L1 or PD-L2 fused to an antibody constant region (e.g., an Fc region of an immunoglobulin sequence). In an embodiment, the PD-1 binding molecule is AMP-224. AMP-224, also known as B7-DCIg, is a PD-L2-Fc fusion soluble receptor described in WO2010/027827 and WO2011/066342.

MDX-1106, also known as MDX-1106-04, ONO-4538, BMS-936558, or nivolumab, is an anti-PD-1 antibody described in WO2006/121168. MK-3475, also known as lambrolizumab (pembrolizumab), is an anti-PD-1 antibody described in WO2009/114335. CT-011, also known as hBAT, hBAT-1, or pidilizumab, is an anti-PD-1 antibody described in WO2009/101611.

In an embodiment, the PD-L1 binding molecule is a PD-L1 binding antagonist, and in particular, is an anti-PD-L1 antibody. Exemplary anti-PD-L1 antibodies include MPDL3280A (atezolizumab), YW243.55.S70, MDX-1105, MEDI4736 (durvalumab), and MSB0010718C (avelumab). Antibody YW243.55.S70 is an anti-PD-L1 antibody described in WO 2010/077634. MDX-1105, also known as BMS-936559, is an anti-PD-L1 antibody described in WO2007/005874. MEDI4736 is an anti-PD-L1 monoclonal antibody described in WO2011/066389 and US2013/034559.

Additional ICIs include ipilimumab (anti-CTLA-4), tremelimumab (anti-CTLA-4), BMS-986016 (anti-LAG-3), urelumab (anti-4-1BB), MSB001078C (anti-4-1BB), TRX51 (anti-GITR), dacetuzumab (anti-CD40), lucatumumab (anti-CD40), SEA-CD40 (anti-CD40), CP-870,893 (anti-CD40), MED16469 (OX40), and MOXR0916 (OX40).

In a specific aspect, the ICI is an anti-PD-1 antibody.

The ICI is conjugated to the platelet cells. In order to conjugate the ICI to the platelet cells, the ICI can be chemically modified with, for example, a bifunctional linker. In an embodiment, the bifunctional linker includes SMCC (succinimidyl-4-(N-maleimidomethyl)cyclohexane-1-caboxylate), MBS (m-maleimidobenzoyl-N-hydroxysuccinimide ester), sulfo-MBS (m-maleimidobenzoyl-N-hydroxysulfosuccinimide ester), GMBS (N-γ-maleimidobutyryloxysuccinimide ester), sulfo-GMBS (N-γ-Maleimidobutyryloxysulfosuccinimide ester, EMCH (N-(8-maleimidocaproic acid) hydrazide), EMCS (N-(ε-maleimidocaproyloxy) succinimide ester), sulfo-EMCS N-(ε-maleimidocaproyloxy) sulfo succinimide ester), PMPI (N-(ρ-maleimidophenyl) isocyanate), SIAB (N-succinimidyl (4-iodoacetyl)aminobenzoate), SMPB (succinimidyl 4-(ρ-maleimidophenyl) butyrate), sulfo-SIAB (N-sulfosuccinimidyl (4-iodoacetyl)aminobenzoate), sulfo-SMCC (sulfosuccinimidyl 4-(N-maleimidomethyl)cyclohexane-1-carboxylate), sulfo-SMBP (sulfo succinimidyl 4-(ρ-maleimidophenyl) butyrate), EDC (1-ethyl-3-(3-dimethylaminopropyl) carbodiimide hydrochloride), or MAL-PEGSCM (maleimide PEG succinimidyl carboxymethyl). In an embodiment, the bifunctional linker comprises SMCC or an NHS ester. SMCC is a hetero-bifunctional linker that contains N-hydroxysuccinimide (NHS) ester and maleimide groups that allow covalent conjugation of amine- and sulfhydryl-containing molecules. NHS esters react with primary amines at pH 7-9 to form amide bonds, while maleimides react with sulfhydryl groups at pH 6.5-7.5 to form stable thioether bonds.

In an aspect, the second component of the treatment regimen is a platelet-membrane coated chemotherapeutic agent nanoparticle (PM-NP).

A “chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, rapamycin (Sirolimus, RAPAMUNE®, Pfizer), lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (such as bullatacin and bullatacinone); a camptothecin (including topotecan and irinotecan); bryostatin; callystatin; CC-1065 (including its adozelesin, carzelesin and bizelesin synthetic analogs); cryptophycins; adrenocorticosteroids (including prednisone and prednisolone); cyproterone acetate; 5α-reductases including finasteride and dutasteride; vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I; dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agents also include anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); aromatase inhibitors that inhibit the enzyme aromatase, which regulates estrogen production in the adrenal glands, such as, for example, 4 (5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); protein kinase inhibitors; lipid kinase inhibitors; antisense oligonucleotides, particularly those which inhibit expression of genes in signaling pathways implicated in aberrant cell proliferation, such as, for example, PKC-alpha, Ralf and H-Ras; ribozymes such as VEGF expression inhibitors (e.g., ANGIOZYME®) and HER2 expression inhibitors; vaccines such as gene therapy vaccines, for example, ALLOVECTIN®, LEUVECTIN®, and VAXID®; PROLEUKIN®, rlL-2; a topoisomerase 1 inhibitor such as LURTOTECAN®; ABARELIX® rmRH; and pharmaceutically acceptable salts, acids and derivatives of any of the above.

Chemotherapeutic agents also include antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories), which is a recombinant, exclusively human-sequence, full-length IgG1 A antibody genetically modified to recognize interleukin-12 p40 protein.

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, all-trans retinoic acid (ATRA), valrubicin, zoledronate, and zoledronic acid, and pharmaceutically acceptable salts thereof.

The chemotherapeutic agent is encapsulated in a biodegradable nanoparticle. As used herein, “biodegradable” materials are those that, when introduced into cells, are broken down by cellular machinery (e.g., enzymatic degradation) or by hydrolysis into components that cells can either reuse or dispose of without significant toxic effects on the cells. In certain aspects, components generated by the breakdown of a biodegradable material do not induce inflammation and/or other adverse effects in vivo. Biodegradable materials may be enzymatically broken down, or broken down by hydrolysis, for example, into their component polymers. Breakdown of biodegradable materials (including, for example, biodegradable polymeric materials) may include hydrolysis of ester bonds, cleavage of urethane linkages, and the like.

Exemplary materials for biodegradable nanoparticles include poly-lactic acid (PLA); poly-D-L-glycolide (PLG); poly-D-L-lactide-co-glycolide (PLGA), poly-alkyl-cyanoacrylate (PCA), poly-8-caprolactone, gelatin, alginate, chitosan, agarose, a polysaccharide, a protein, or a combination thereof. Biodegradable nanoparticles can be made by techniques known in the art such as solvent evaporation, spontaneous emulsification, nanoprecipitation, salting out, polymerization, or ionic gelation of hydrophilic polymers, for example.

In an aspect, the biodegradable nanoparticle comprises a polysaccharide. The term “polysaccharide” refers to a polymer of sugars. Polysaccharide nanoparticles described herein may be made of polysaccharides such as dextran, amylose, amylopectin, glycogen, cellulose, arabonixylan, and/or pectin. In certain embodiments, the polysaccharide is dextran. Dextran is a complex, branched glucan (a polysaccharide made of many glucose molecules) composed of chains of varying lengths (from 3-2000 kilodaltons). The straight-chain comprises alpha-1,6-glycosidic linkages between glucose molecules, while branching begins at alpha-1,3 linkages. In some embodiments, dextran nanoparticles are comprised of carboxymethyl dextran.

The polysaccharides that make up the nanoparticles can have a range of molecular weights such as 1 kDa to 1 million kDa (e.g., 1-10 kDa, 10-100 kDa, 100-1000 kDa, or 1000-1,000,000 kDa). The polysaccharide nanoparticles can have an average diameter in a range of 1 nm-500 nm (e.g., 1-10 nm, 10-25 nm, 25-50 nm, 50-100 nm, or 100-500 nm). The polysaccharide nanoparticles may be relatively monodisperse (e.g., diameters of particles all within a range of 10 nm or less of each other) or more polydisperse.

The NP encapsulating the chemotherapeutic agent is then coated with a platelet membrane. Platelet membranes can be isolated from platelets using ultrasonication. The platelet membranes and the NPs are then mixed and sonicated to form the PM-NPs.

The first component and the second component are preferably administered as separate pharmaceutical compositions. Injectable compositions, for example, injectable aqueous or oleaginous suspensions may be formulated according to the known art using dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween™ 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

In an aspect, the first component, the second component or both is locally or systemically administered. In the treatment of solid tumors, local administration can include delivery at or near the site of the tumor. One type of local delivery is peritumoral injection (p.t.) delivery, or intratumor injection.

Systemic administration is administration through the circulatory system, such as via intravenous injection.

In an aspect, the period of time is dependent upon the mode of administration. When the fusion polypeptide is administered by local administration, the period of time is 15 minutes to 4 hours. When the fusion polypeptide is administered by systemic administration, the period of time is 60 minutes to 4 hours.

Exemplary solid tumors for treatment with the methods described herein include bladder, breast, cervix, colon, rectal, endometrial, kidney, oral, liver, lung, melanoma, non-small cell lung cancer, ovarian, pancreatic, prostate, sarcoma, small cell lung cancer, and thyroid, for example. Solid tumors also include metastatic tumors.

The invention is further illustrated by the following non-limiting examples.

### EXAMPLES

### Materials and Methods

Antibodies, cells, and mice: The antibodies, including GoInVivo™ Purified anti-mouse CD279 (PD-1) antibody (Clone #: RMP1-14; Category #: 114114), APC-anti-mouse CD3 (Clone #: 17A2, Category #: 100236), PE-anti-mouse CD8a (Clone #: 53-6.7, Category #: 100708), FITC-anti-mouse IFNγ (Clone #: XMG1.2, Category #: 505806) were purchased from BioLegend. All antibody dilutions were performed according to the manufacturer's guidance. The flow cytometry data were analyzed by FlowJo v10 software.

The mouse CT26 colon cancer cells were obtained from ATCC, and Luc-CT26 cells were purchased from Imanis Life Sciences Inc. Luciferase-tagged CT26 cells were used for in vivo bioluminescence imaging. TM00096 breast cancer model was obtained from the Jackson Laboratory. The Balb/C mice (Male, aged 6-8 weeks) and nude mice (Female, aged 6-8 weeks) were purchased from the Jackson laboratory. The Rag2-II2rg double knockout mice (R2G2®) mice (Female, aged 6-8 weeks) were obtained from ENVIGO. The animal study protocol was approved by the Institutional Animal Care and Use Committee (IACUC) at the University of Wisconsin-Madison.

Expression, purification and characterization of tTF and tTF-RGD: Full-length human tissue factor tTF) amino acid sequence was obtained from Uniprot (P13726). The cDNA coding truncated tissue factor (tTF) containing amino acid 33-250 and the C-terminal extension (tTF-RGD) were synthesized by Genscript, then inserted into pET30a (+) vector using BamHI and NcoI to generate tTF and tTF-RGD expression vector pET30a (+)-tTF and pET30a (+)-tTF-RGD. The tTF and tTF-RGD plasmid were introduced in Escherichia coli BL21 (DE3) (Invitrogen), and the protein was purified with Nickle-nitrilotriacetic acid (Ni-NTA) (Qiagen).

tTF: Primer 1:
SEQ ID NO: 10)
5'-CATGCCATGGGATCAGGCACTACAAATACTGTG-3'
Primer 2:
(SEQ ID NO: 11)
5'-CGGGATCCTATTATCTGAATTCCCCTTTC-3'
tTF-RGD: Primer 1:
(SEQ ID NO: 12)
5'-CATGCCATGGGATCAGGCACTACAAATACTGTG-3'
Primer 3:
(SEQ ID NO: 13)
5'-CGGGATCCTATTATGGAGAATCACCTCTTCCTCTGAATTCCCC-3'

A single colony of E. coli BL21 (DE3) introduced with the vectors was selected and cultivated in the liquid LB medium containing kanamycin (50 μg/mL). When the OD₆₀₀ of bacteria solution reached approximately 0.6, 0.8 mM IPTG was added to induce the over-expression of the fusion proteins. After approximately 6 h, the cells were harvested and centrifuged at 2,109 g for 30 min at 4° C. Cells were suspended in lysis buffer and homogenized with a tissue grinder homogenizer. The cells were then incubated for 30 min at room temperature and centrifuged at 12,000 g for 30 min at 4° C. The obtained pellet was resuspended in washing buffer, followed by homogenization with a tissue grinder and sonicating with the sonicate parameter (sonication time: 5 sec; interval time: 5 sec, totally 30 min). The mixture was centrifuged at 12,000 g for 30 min at 4° C., and the supernatant was discarded. The procedure was repeated until the supernatant was clean. tTF and tTF-RGD proteins were extracted from washed pellets with a Ni NTA column. The proteins in elution buffers were refolded by gradient dialysis against GuCl dialysis buffers containing a decreasing concentration of GuCl. The purity of tTF and tTF-RGD was tested using SDS-PAGE, and the concentration was determined with a BCA kit. The final proteins (>96% purity) were stored at −80° C.

The FX activation activity was tested using a modified procedure based on a previous report in the art. Briefly, to each well of a black 96-well plate was added 100 μL of BSA, tTF, and tTF-RGD with different concentrations in Tris-buffered saline containing 0.1% BSA. Then, 10 μL of FVIIa solution (Haematologic Technologies, Inc.) was added, followed by the rapid addition of 10 μL of 50 nM CaCl₂) and 500 μM phospholipids. After 5 min of incubation at room temperature, FX (Haematologic Technologies, Inc.) was added to reach a final concentration of 30 nM and reacted for an additional 5 min. The reaction was quenched by addition of 20 μL of 100 mmol/L EDTA solution before 10 μL of 6-amino-1-naphthalenesulfonamide-based (ANSN) fluorogenic substrate (10 mM) (Haematologic Technologies, Inc.) was added to the mixture. Three minutes later, the fluorescence intensities were measured with a microplate reader (excitation=352 nm, emission=470 nm).

Tumor vasculature-targeting and coagulation initiating ability tTF-RGD: The tumor vasculature-targeting ability of tTF-RGD was investigated using IVIS. Briefly, tTF and tTF-RGD were labeled with sulfo-Cy5.5-NHS ester and dialyzed against PBS at 4° C. to remove unreacted dyes. Balb/C mice (6-8 weeks, male) were inoculated with 1×10⁶ CT26 cells. When the tumor volumes reached 100-150 mm³ (calculated as length×width²×0.5), Cy5.5-labeled tTF or tTF-RGD were injected at the dose of 1.25 mg/kg via i.v. route to mice bearing CT26 tumors. The biodistribution of tTF or tTF-RGD was observed at different time points. At 24 h, the mice were euthanized, and tumors were collected and visualized ex vivo. The fluorescence was analyzed using Living Image Software v.4.3.1 (Perkin Elmer).

To assess the tumor vasculature-coagulation triggered by tTF-RGD, fibrinogen was labeled with sulfo-Cy5.5-NHS ester and dialyzed against PBS to remove unreacted dyes. BSA, tTF or tTF-RGD were injected at the dose of 1.25 mg/kg via i.v. route to mice bearing CT26 tumors, followed by i.v. injection of 1 nmol of fibrinogen. The biodistribution of fibrinogen was observed at different time points. At 24 h, the mice were euthanized, and tumors were collected and visualized ex vivo.

The coagulation initiation capability of tTF-RGD was investigated by treating CT26 tumor-bearing mice with 1.25 mg/kg of BSA, tTF, and tTF-RGD via intratumoral injection. Twenty-four hours later, the tumors were collected and subjected to H&E staining. To characterize the coagulation triggered by a lower dose of the tTF-RGD, 0.4 mg/kg of BSA, tTF, and tTF-RGD were administered into CT26 tumor-bearing mice via p.t. injection. Furthermore, mice bearing CT26 tumors were also treated with BSA, tTF and tTF-RGD (1.25 mg/kg) via i.v. injection every other day for 3 times in total. Twenty-four hours post injection of the last dose, the tumors and other major organs were collected and subjected to H&E staining.

Preparation and characterization of P-aPD-1: The platelets were isolated from whole mouse blood by gradient centrifugation. The collected blood was centrifuged for 20 min at 100 g twice to remove red blood cells, followed by centrifugation for 20 min at 1,500 g to isolate platelets. The platelets were resuspended in PBS containing 1 μM PGE1. To prepare P-aPD-1, aPD-1 antibodies and Sulfo-SMCC linkers were mixed at a molar ratio of 1:1.2 and stirred at 4° C. Two hours later, the solution was centrifuged in ultrafiltration tubes (3000 kDa MWCO) at 4° C. to remove the unbound linkers. The SMCC-aPD-1 was reacted with Traut's reagent-treated platelets at room temperature for 1 h, followed by removing the excess antibodies using centrifugation at 1,500 g for 20 min. The amount of the conjugated aPD-1 was measured using Rat IgG total ELISA Kit after applying 0.1% Triton™M_X100 solution to release the antibody under sonication.

To verify the conjugation of aPD-1 and platelets, aPD-1 stained with FITC, and platelets stained with WGA 594 were used to prepare P-aPD-1 and were observed under the confocal microscope. To study the thrombin-triggered activation of platelets, Rhodamine B-labeled P-aPD-1 or platelets were cultured in a complete medium supplemented with thrombin (0.5 U/mL) and then visualized using the confocal microscope. To study the collagen binding capability, confocal dishes were coated with 1.0 mg/mL of collagen (Sigma) overnight at 4° C. 2% BSA in PBS was used to block the coated or uncoated confocal dishes. Platelets or P-aPD-1 labeled with NHS-Rhodamine B were then incubated in the dishes for 5 min. The dishes were washed with PBS to remove the unbound platelets or P-aPD-1, and then visualized using the confocal microscope.

Recruitment of P-aPD-1 by tTF-RGD treatment: The recruitment of P-aPD-1 to tumor sites by tTF-RGD-initiated coagulation was studied using IVIS. P-aPD-1 was reacted with sulfo-Cy5.5-NHS ester at room temperature for 1 h and washed three times with PBS to remove unreacted dyes. For the local treating strategy, BSA, tTF, and tTF-RGD were given at the dose of 0.4 mg/kg via p.t. injection to mice bearing CT26 tumors, followed by i.v. injection of approximately 1×10⁸ P-aPD-1 (aPD-1:1 mg/kg). For the systemic treatment strategy, BSA, tTF, and tTF-RGD were given at the dose 1.25 mg/kg via i.v. injection to mice bearing CT26 tumors, followed by i.v. injection of approximately 1×10⁸ P-aPD-1 (aPD-1:1 mg/kg). The biodistribution of P-aPD-1 was observed at different time points. At 24 h, the mice were euthanized, and tumors were collected and visualized ex vivo.

Anti-tumor efficacy of tTF-RGD+P-aPD-1: To establish a subcutaneous CT26 colon cancer model, Balb/C mice (6-8 weeks, male) were subcutaneously inoculated with 1×10⁶ CT26 cells on the right flank. When the tumor volume reached100-150 mm³, the mice were received different treatments, including saline, aPD-1, tTF-RGD, P-aPD-1, tTF+P-aPD-1 and tTF-RGD+P-aPD-1. For the local treating strategy, tTF or tTF-RGD (0.4 mg/kg) was firstly given by p.t. injection. Half an hour later, P-aPD-1 was administered via i.v. injection at the dose of 1 mg/kg. The treatment was conducted every other day for 3 times in total. For the systemic treating strategy, tTF or tTF-RGD (1.25 mg/kg) was first given by i.v. injection. One hour later, P-aPD-1 was administered via i.v. injection at the dose of 0.3 mg/kg. The treatment was conducted every other day for 3 times in total. The antibodies used in other treatment groups are also given at an equivalent dose. The tumor growth (calculated as: length×width²×0.5) and body weight were monitored every other day. Mice were euthanized once the tumor volume exceeded 2 cm³.

Tumor tissues were collected on day 12 after the first treatment, digested with collagenase, and passed through cell strainers with pore sizes of 40 μm to obtain single-cell suspension. The cells were collected by centrifugation, and the supernatant was used for the detection of cytokines. The cells were resuspended in PBS and stained with APC-anti-mouse CD3, PE-anti-mouse CD8a, and analyzed using flow cytometry. The staining of FITC-anti-mouse IFNγ was conducted according to the intracellular flow cytometry staining protocol.

The tumor tissue supernatant after centrifugation was used for the detection of IFN-γ and TNF-α with the corresponding ELISA kits. The fold change was calculated for cytokine levels after different treatments relative to the saline group. IL-1a, IL-1B, IL-3, IL-27, IFN-β, GM-CSF were tested using LEGENDplex™ Mouse Cytokine Panel 2 (BioLegend, 740134) according to the guidance of manufacturers.

Synthesis of dextran NP and preparation and characterization of PM-NP: Modified dextran was synthesized according to protocols in the art. Briefly, 1.0 g of dextran (Mn approximately 9-11 kDa) was dissolved in 10 mL DMSO. Then, 15.6 mg of pyridinium P-toluenesulfonate and 2-ethoxypropene (4.16 mL) were added to the solution and stirred for 30 min at room temperature. Afterwards, 1 mL of triethylamine was added to quench the reaction. The precipitated mixture was washed in basic water (pH approximately 8) before lyophilization.

Dextran NPs were prepared using a single-emulsion method. Briefly, 10 mg mDex and 0.5 mg PTX were dissolved in 2 mL dichloromethane (DCM) and were gently added to 4 mL 3% poly(vinyl alcohol) (PVA) solution. The mixture was subjected to sonication for 2 min (% Amplification, 35%, 2s on and 2s off circles) before being dispersed in 20 mL 0.3% PVA solution. The solution was stirred to evaporate the DCM. NPs were collected by centrifugation and washed twice to remove the unencapsulated drugs. To load the NIR dyes into NPs, 10 mg modified dextran and 0.1 mg DiR were used to prepare DiR-loaded nanoparticles with the same procedures as described above.

To prepare PM-NP, first, platelets isolated from mouse blood were diluted with sterile ddH₂O and disrupted with ultrasonication. The platelet membranes were centrifuged at 4,500 g for 5 min and washed twice with sterile ddH₂O. The pH of the solution was adjusted to 7. The platelet membranes and NPs were sonicated together for 30 seconds, % A 35%, 2s on and 2s off circles before stirring for 3 hours at room temperature. The formed PM-NP was centrifuged at 21,900 g for 30 min and washed with ddH₂O. The nanoparticles were diluted in sterile saline for in vivo study. The morphologies of NPs and PM-NPs were observed under TEM and their size was tested by DLS. The drug encapsulation efficiency was determined by HPLC.

Establishment of the PDX model: To establish the PDX model, TM00096 tumor samples were first propagated on Rag2-II2rg double knockout immunodeficient mice (R2G2® mice, 6-8 weeks, female) and then passaged to nude mice (6-8 weeks, female). Briefly, tumor tissues from R2G2® mice with TM00096 tumors were cut into small pieces, dispersed in the 1:1 mixture of DMEM medium and Matrigel® supplemented with FBS and antibiotics. The tiny tumor tissues were mixed well and placed on ice packs before being injected into the right flank of the nude mice using an 18-gauge needle (100 μL/mouse).

Recruitment of PM-NP by tTF-RGD-triggered coagulation: The recruitment of PM-NP to tumor sites by tTF-RGD-initiated coagulation was studied using IVIS. NPs were loaded with DiR and coated with platelet membranes. The PM-NP was washed three times with PBS to remove unreacted dyes. tTF or tTF-RGD was given at the dose of 1.25 mg/kg via i.v. injection to mice bearing PDX breast tumors. One hour later, 2 mg of NPs and PM-NPs were administered via i.v. injection. The biodistribution of NP and PM-NP was observed at different time points. At 24 h, the mice were euthanized, and tumors were collected and visualized ex vivo.

Anti-tumor efficacy of tTF-RGD+PM-NP: The PDX breast tumor model was established as described in the art. When the tumor volumes reached 100-150 mm³, the mice were randomly grouped and received different treatments, including saline, PM-NP/PTX, tTF+PM-NP/PTX and tTF-RGD+PM-NP/PTX. tTF or tTF-RGD (1.25 mg/kg) was first given by i.v. injection. One hour later, PM-NP/PTX was administered via i.v. injection at the PTX dose of 2.5 mg/kg. The treatment was conducted every other day for 3 times in total. The tumor volume (calculated as: length×width²×0.5) and body weight change were monitored. Mice were euthanized once the tumor volume exceeded 1.5 cm³.

Statistical analysis: All the results are shown as mean±SEM. Statistical analysis was performed using GraphPad Prism (version 9) and ANOVA was used to compare multiple groups statistically. Survival studies were analyzed using Log-rank test. A P value lower than 0.05 (*P<0.05) was defined as statistically significant; *P<0.05, **P<0.01, ***P<0.001.

### Example 1: Synthesis and Characterization of Fusion Protein tTF-RGD

Compared with TF, the Factor X (FX) activation activity of tTF, a truncated TF that that lacks a transmembrane domain, decreases by five orders of magnitude as reported in the art. To restore the coagulation initiating capability of tTF, an RGD motif was adopted herein as a transmembrane domain to redirect the tTF to tumor vasculature, which is expected to trigger tumor-selective thrombosis with an improved in vivo biosafety profile compared to TF. The tumor-targeted chimeric protein tTF-RGD was generated by fusing the RGD motif to the extracellular domain of TF. The purity of tTF and tTF-RGD was verified by SDS-PAGE (FIG. 1B), which showed over 96% purity for both tTF and tTF-RGD. The activity of tTF and tTF-RGD was validated by FX activation assay. As presented in FIG. 1C, both tTF and tTF-RGD exhibited a dose-dependent FX activation activity in the presence of factor VII (a) and Ca²⁺ ions. Moreover, the capability of tTF-RGD to initiate coagulation in vivo was first examined by direct intratumoral injection (FIG. 1D). After 24 h post-injection, the tumors were collected, and severe coagulation was observed in tumors after tTF-RGD treatment, whereas minimal to modest coagulation was observed in tTF-treated tumors. Considering the limited application of intratumoral injection in the clinical treatment of tumors, two strategies may be adopted for in vivo application in which tTF-RGD will be administered, specifically via p.t. (local) or i.v. (systemic) routes. Data not shown shows that there was a significant hemorrhage and thrombus formation in the tumors after intratumoral injection of tTF-RGD with a relatively high concentration (1.25 mg/kg), which may impede the infiltration of immune cells and therapeutic drugs. Thus, in the p.t. injection, the administration dose of tTF-RGD was decreased to 0.4 mg/kg, which effectively triggered obvious coagulation without severe hemorrhage. The intravenous injection of tTF-RGD will be further tested for efficacy in initiating tumor-selective thrombus formation. In data not shown, 1.25 mg/kg of tTF-RGD every other day for three times injected through i.v. routes did not induce severe hemorrhage but could trigger thrombosis. In addition, the efficacy of tTF and tTF-RGD were compared, with bovine serum albumin (BSA) serving as the scrambled control group. As shown in FIG. 1E, tTF-RGD initiated apparent coagulation while this phenomenon was not observed after both tTF and BSA treatments. Besides, the hematoxylin and eosin (H&E) staining suggests that there was no obvious hemorrhage or thrombus in other major organs after 3 doses of i.v. injected tTF-RGD, demonstrating the tumor-selective thrombosis and great in vivo biosafety profile of tTF-RGD (data not shown). Both tTF and BSA did not trigger coagulation in any organs, including tumor tissues. Without being held to theory, this is attributed to the lack of targeting ability and the loss of the transmembrane domain of tTF.

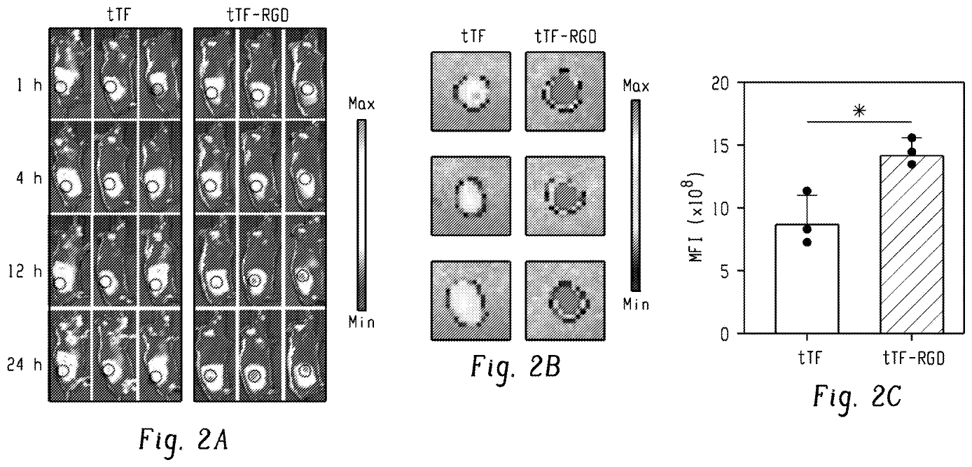

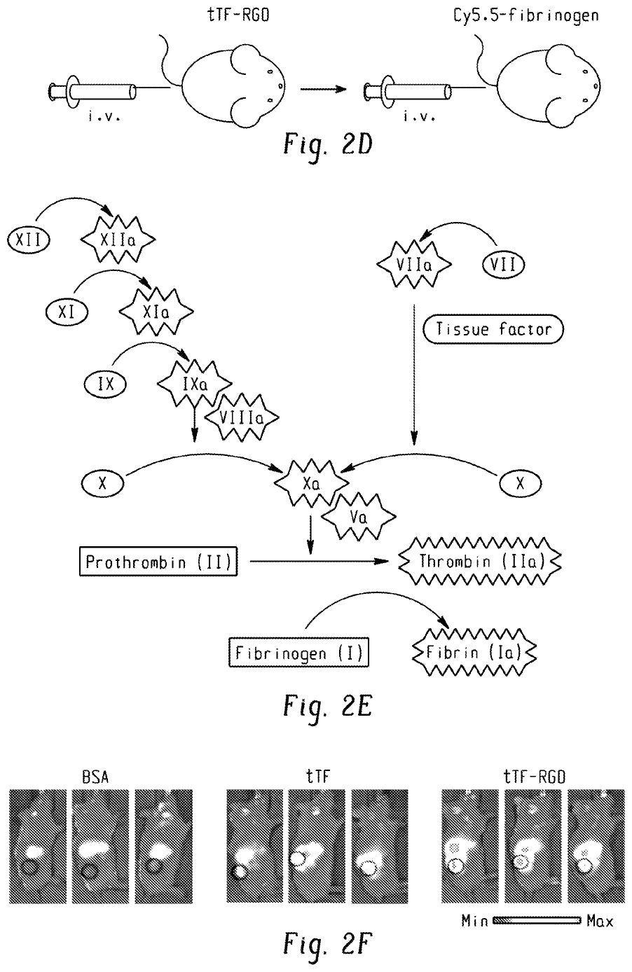

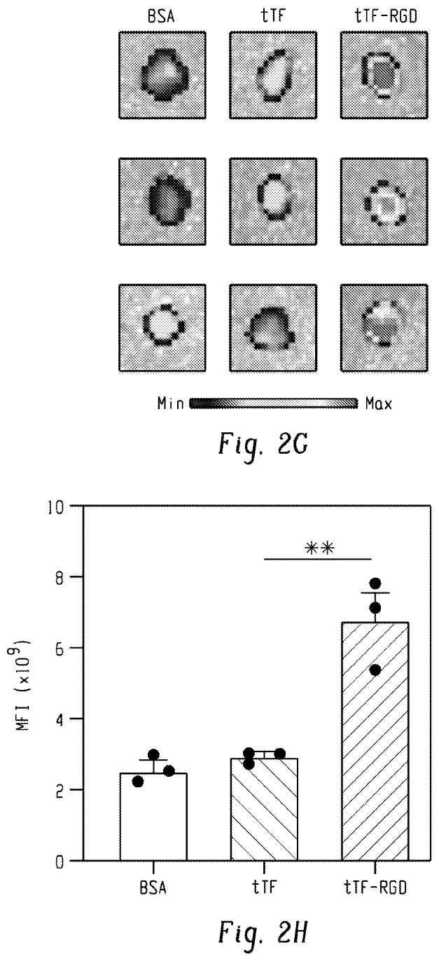

Therefore, the tumor-targeting ability of tTF-RGD was examined. Mice bearing subcutaneous CT26 tumors were injected with Cy5.5-labeled tTF and tTF-RGD via the i.v. route and visualized using IVIS. As shown in FIG. 2A, tTF-RGD group exhibited much stronger tumor targeting ability than tTF, as evidenced by the brighter fluorescence signals at the tumor site during the time-course of the treatment. At 24 h post-administration, the tumor tissues were collected and visualized ex vivo, which showed an increased accumulation of tTF-RGD than tTF (FIG. 2B). Quantitatively, the mean fluorescence intensity (MFI) of tumor tissues in tTF-RGD group was 1.62-fold higher than that in the tTF group (FIG. 2C), substantiating the tumor-targeting ability of tTF-RGD, which was attributed to the RGD binding affinity towards the integrin receptors on the angiogenic blood vessels in the tumor site. Next, to investigate the capabilities of tTF-RGD in initiating coagulation, fibrinogen (the precursor to fibrin) was injected via the i.v. route right after i.v. injection of BSA or tTF or tTF-RGD (FIG. 2D). TF naturally serves as an initiator of blood coagulation by forming the TF: FVIIa complex, which drives the activation of FX and the subsequent transformation of prothrombin to thrombin (FIG. 2E). Thrombin then converts soluble fibrinogen to insoluble fibrin, which can form clots together with the activated platelets. Without being held to theory, it is believed that the fusion protein tTF-RGD can also initiate the coagulation cascade in a tumor-specific manner. Given that tissue coagulation contributes to the conversion of fibrinogen to fibrin and subsequent deposition of fibrinogen, Cy5.5-labeled fibrinogen was used to characterize the coagulation process in tumors. At 24 h post injections of tTF-RGD and Cy5.5-fibrinogen, strong signals were observed in tumors treated with tTF-RGD+fibrinogen group, while fibrinogen did not preferentially accumulate in tumors after BSA or tTF treatment (FIG. 2F, G). Compared with tTF group, the MFI in tumors after tTF-RGD treatment increased by 2.37-fold (FIG. 2H). The results substantiated that tTF-RGD could effectively induce coagulation to attract fibrinogen at tumor sites.

### Example 2: Recruitment of Platelets to the Tumor Site by tTF-RGD

After validating that tTF-RGD is capable of selectively triggering coagulation at tumor sites, it was investigated if administrated P-aPD-1 could take advantage of this process to be actively recruited to the tumor site. Platelets are quick responders to vascular injury and hemorrhage. Besides, after the initiation of the coagulation cascade, the generated thrombin and other signal molecules can activate platelets to aggregate with fibrin to form a platelet plug locally and build up the thrombus. Therefore, it was anticipated that P-aPD-1 could also selectively accumulate at the tumor sites during the tTF-RGD-initiated coagulation. The P-aPD-1 was prepared by conjugating amine groups on the antibodies to the thiol groups on the platelet surface with Sulfo-SMCC linkers. The overlap between the red signals from Wheat Germ Agglutinin (WGA) 594-labeled platelets and the green signals from fluorescein isothiocyanate (FITC)-labeled antibodies was observed under the confocal microscope, demonstrating the successful modification of aPD-1 on the platelets (data not shown). The bio-functionalities of P-aPD-1 were studied by thrombin-triggered activation test (data not shown) and collagen binding assay (data not shown). The thrombin-triggered activation of platelets was not altered after aPD-1 decoration, as evidenced by the clot formation in both P-aPD-1 and naïve platelets. In addition, there was no significant difference in the collagen-binding capability of P-aPD-1 in comparison with naïve platelets. These properties demonstrated that P-aPD-1 can still respond to coagulation-related biological signals aPD-1.

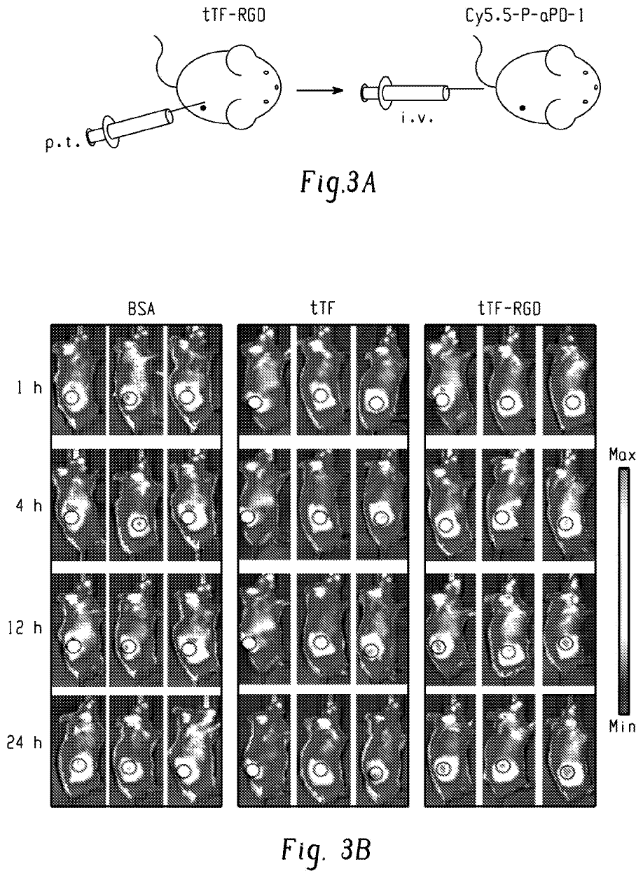

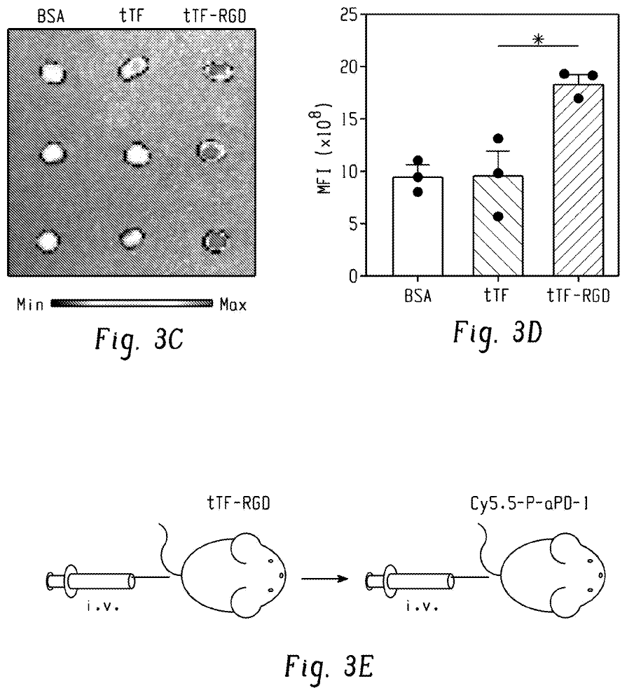

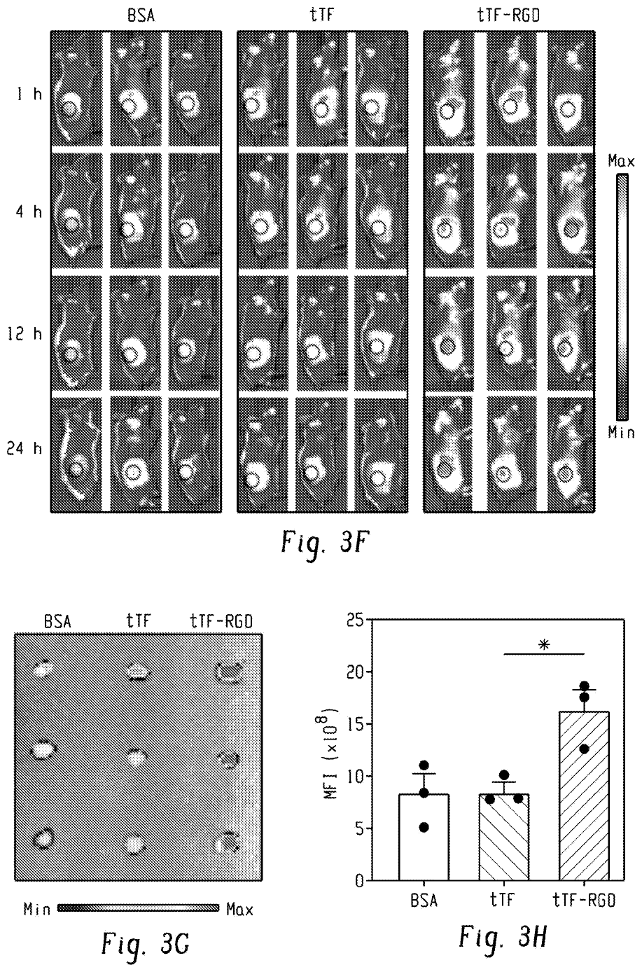

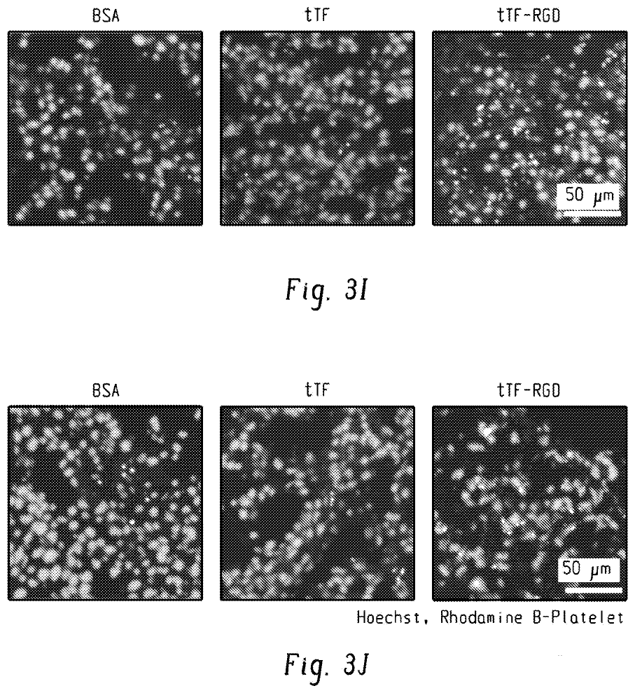

Next, it was determined if the in vivo injection of tTF-RGD can recruit platelets to the tumor sites. Mice bearing CT26 tumors were injected with BSA or tTF or tTF-RGD via p.t. injection, followed by i.v. injection of Cy5.5-labeled P-aPD-1 (FIG. 3A). In the tTF-RGD treatment group, P-aPD-1 showed a strong tendency to accumulate at the tumor site from 4 h post injection (FIG. 3B). At 24 h, there was an obvious higher accumulation of P-aPD-1 in the treatment tTF-RGD group than that in either BSA or tTF treatment groups. The ex vivo quantification of tumor tissues demonstrated a 1.87-fold increase in the fluorescence signals of P-aPD-1 in tTF-RGD groups than that in tTF group, substantiating that p.t. injection of tTF-RGD can attract P-aPD-1 to tumor site by triggering tumor-selective thrombus formation (FIG. 3C, D).

Having verified that tTF-RGD can target the tumor area and local delivery of tTF-RGD can attract platelets to the tumor area, it was further tested if i.v. injection of tTF-RGD can also recruit P-aPD-1 to the tumor sites. Cy5.5-labeled P-aPD-1 was given via the i.v. route after the i.v. injection of tTF-RGD (FIG. 3E). Consistent with the previous finding for p.t. injection of tTF-RGD, intravenous injection of tTF-RGD also induced increased distribution of P-aPD-1 at the tumor sites compared to tTF and BSA treatments during the time-course of IVIS monitoring (FIG. 3F), leading to a 1.89-fold increase in the accumulation of P-aPD-1 in the tumor tissues in the tTF-RGD group than that in tTF group at 24 h post-administration (FIG. 3G, H). P-aPD-1 was further labeled with Rhodamine B and the distribution of platelets was examined in tumors resected from mice that received the treatment of the local and systemic strategies with tTF-RGD. More fluorescence signals were observed in tumor tissues after tTF-RGD treatment either through p.t. injection (FIG. 3I) or i.v. injection (FIG. 3J) compared to BSA and tTF treatments (FIGS. 3I and J). Collectively, these results substantiated that either p.t. or i.v. injection of tTF-RGD can actively recruit P-aPD-1 to the tumor site by selectively triggering thrombosis within tumor tissue.

### Example 3: In Vivo Anti-Tumor Efficacy of tTF-RGD+P-aPD-1 in a CT26 Model

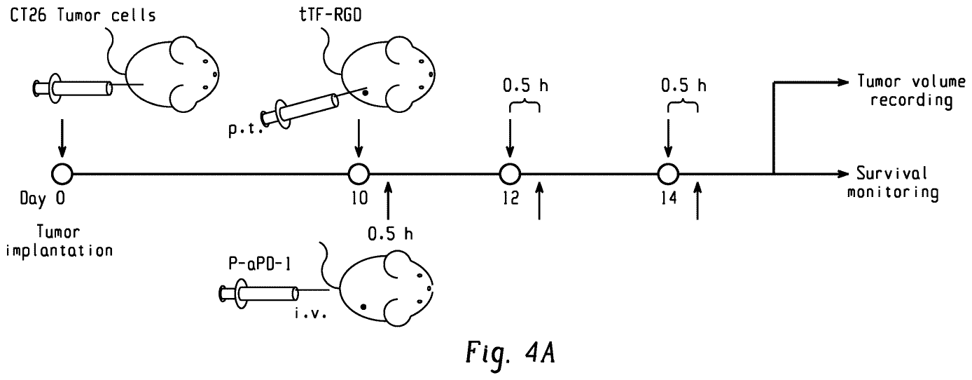

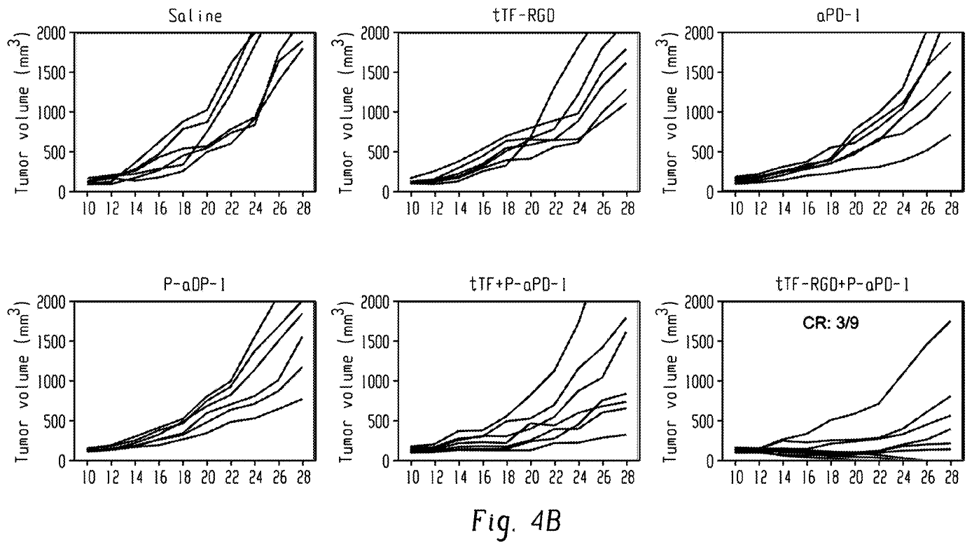

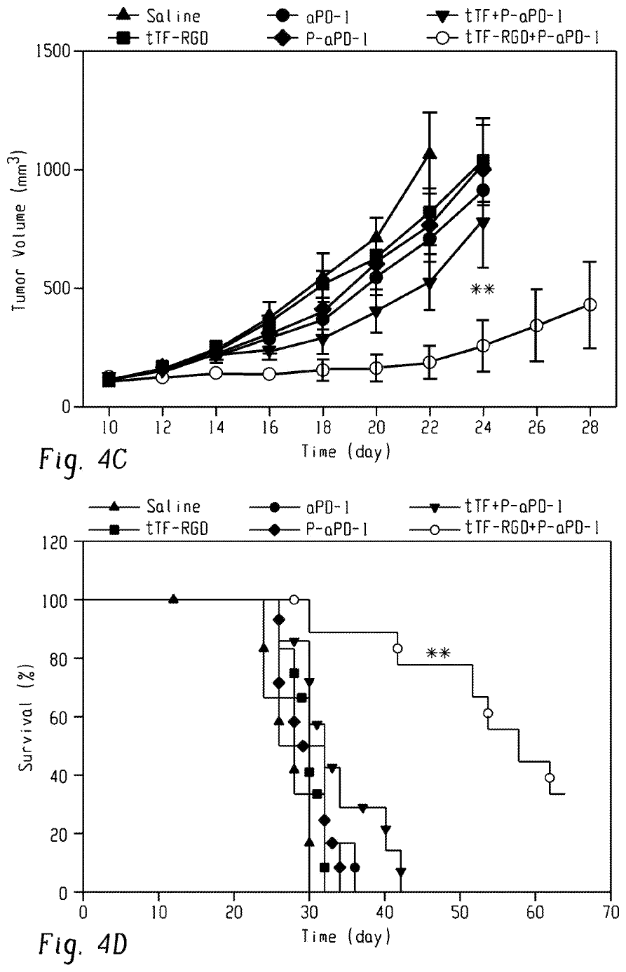

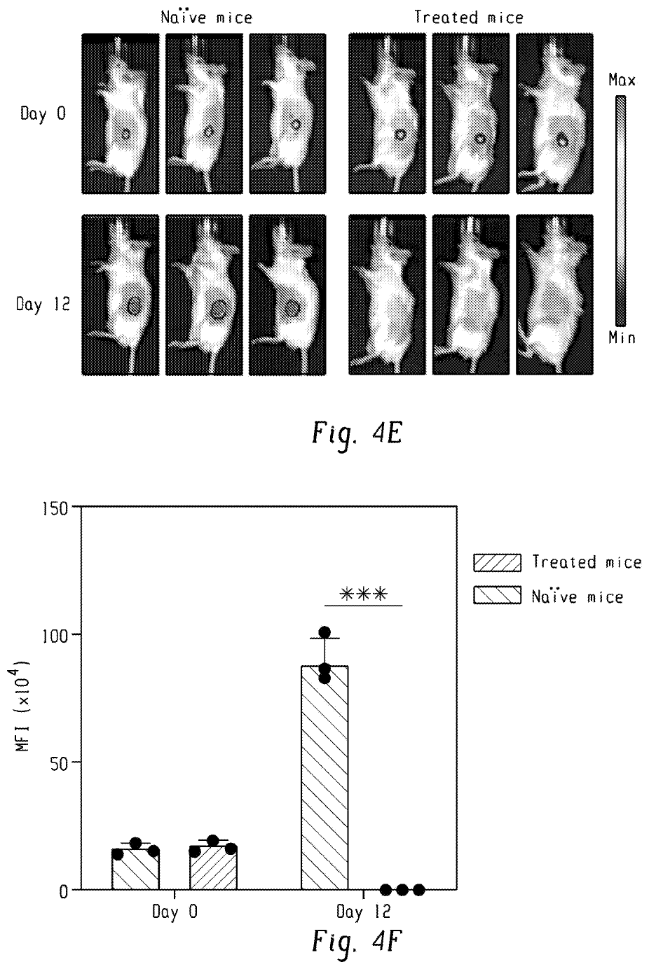

To study the anti-tumor efficacy of tTF-RGD+P-aPD-1 in vivo, a subcutaneous CT26 tumor model was established. When the tumor volume reached 100-150 mm³, the mice were administered different treatments, including saline, tTF-RGD, aPD-1, P-aPD-1, tTF+P-aPD-1, and tTF-RGD+P-aPD-1. Firstly, the therapeutic effect of the local treatment strategy was evaluated, which leveraged p.t. injection of tTF-RGD to attract P-aPD-1 to the tumor site. After 0.5 h post p.t. injection of tTF-RGD at the dose of 0.4 mg/kg, P-aPD-1 at the aPD-1dose of 1 mg/kg was given by i.v. injection (FIG. 4A). As shown in FIG. 4B, C, tTF-RGD only showed a negligible tumor-inhibiting activity against CT26 tumors. In addition, aPD-1 treatment strategies, including free aPD-1, P-aPD-1 and tTF+P-aPD-1, resulted in modest delayed tumor growth inhibition efficacy, leading to slightly increased survival time of CT26 tumor bearing-mice. As a comparison, tTF-RGD+P-aPD-1 exhibited the best tumor inhibition efficacy among all groups, which showed a significantly smaller tumor size than that in other treatment groups at day 24 (FIG. 4B, C). Moreover, tTF-RGD+P-aPD-1 treatment strikingly prolonged the survival time of the mice to a median survival time of 58 days compared to saline (27 days), tTF-RGD (30 days), aPD-1 (31 days), P-aPD-1 (30 days), tTF+P-aPD-1 (32 days) (FIG. 4D). The body weight of the mice was monitored, and no decrease in body weight after the treatment was observed, which demonstrated the in vivo biosafety profile of (data not shown). Notably, 3 out of 9 mice in the tTF-RGD+P-aPD-1 treatment group became tumor free. To substantiate the immune-memory response after tTF-RGD+P-aPD-1 treatment, these tumor-free mice were re-challenged with 1×10⁶ CT26-luc cells at day 70. Compared to the control naïve mice with progressive tumor growth, the treated mice did not develop visible tumors, and no obvious luciferase signal at the injection site was observed in bioluminescence images 12 days post the re-challenge (FIG. 4E, F) and later time points.

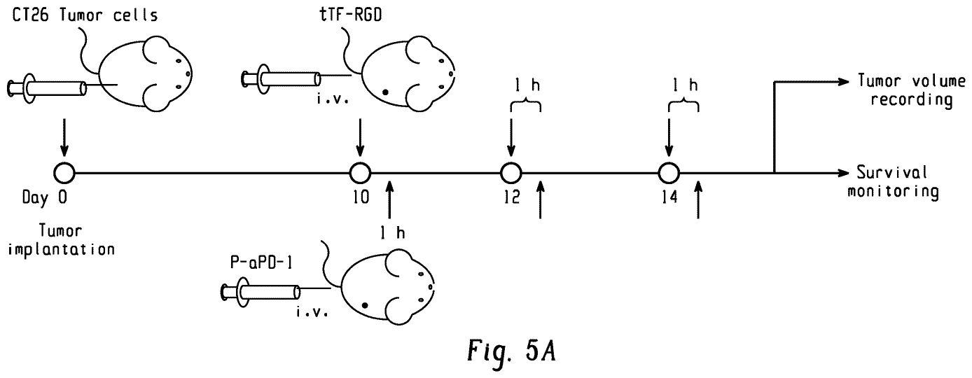

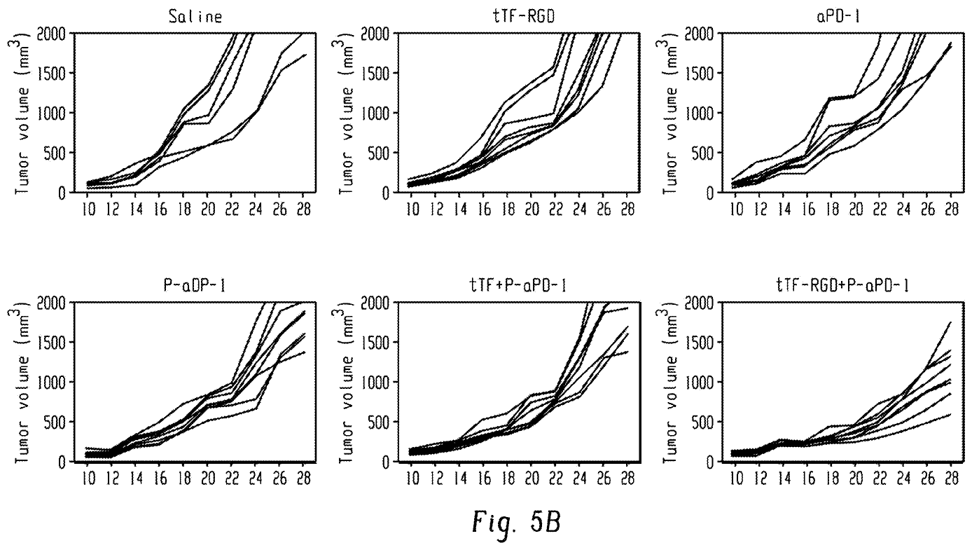

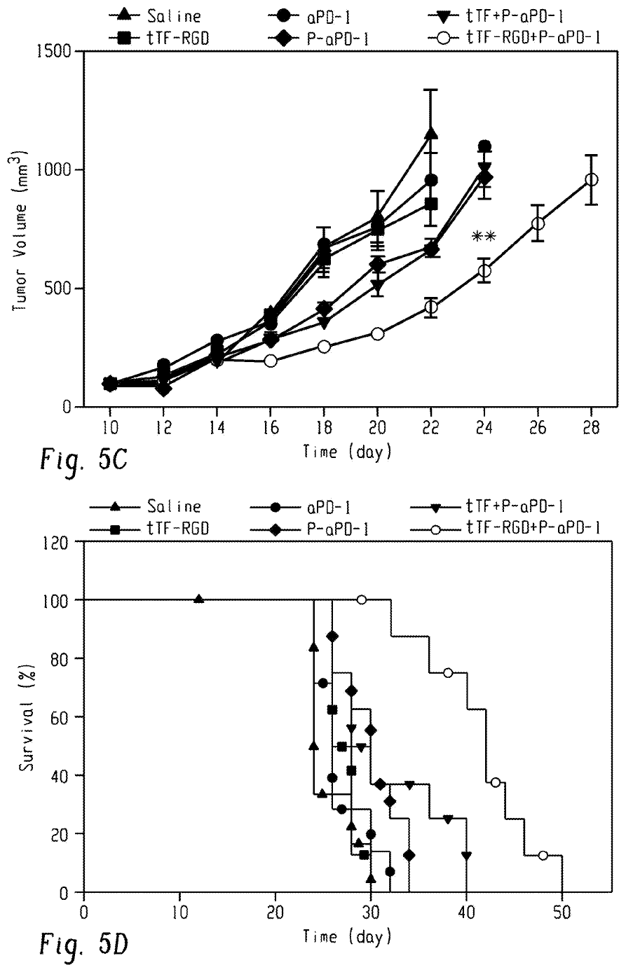

Next, the therapeutic effect of i.v. injected tTF-RGD+i.v. injected P-aPD-1 was tested. Encouraged by the potent treatment efficacy of p.t. tTF-RGD+i.v. P-aPD-1, the dose of aPD-1 was reduced to 0.3 mg/kg, which is a very low dose with which systemically injected free antibodies almost show no effect. After 1 h post i.v. injection of tTF-RGD (1.25 mg/kg), P-aPD-1 was administered by i.v. injection (FIG. 5A). tTF-RGD+P-aPD-1 showed the best performance in inhibiting tumor growth compared to all other treatment groups, in which the average tumor sizes in the tTF-RGD+P-aPD-1 treatment groups are significantly smaller than those in other treatment groups on day 24 (FIG. 5B, C). tTF-RGD+P-aPD-1 treatment prolonged the median survival time of the mice from 24 days (saline group) to 42 days, which is remarkably better than other groups (tTF-RGD: 27 days; aPD-1:26 days; P-aPD-1:30 days; tTF+P-aPD-1:29 days) (FIG. 4D). In addition, no body weight loss was observed after all treatments, evidencing the safety profiles (data not shown).

### Example 4: tTF-RGD+P-aPD-1 Treatment Enhanced T Cell Infiltration

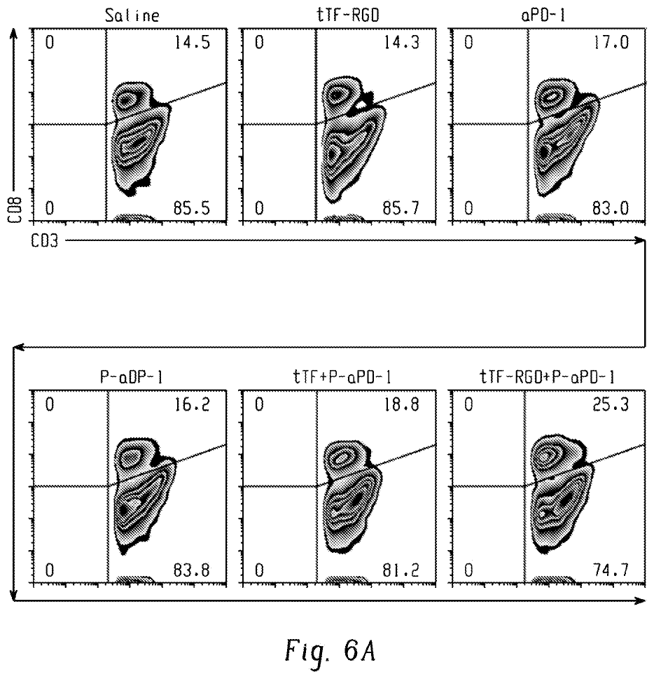

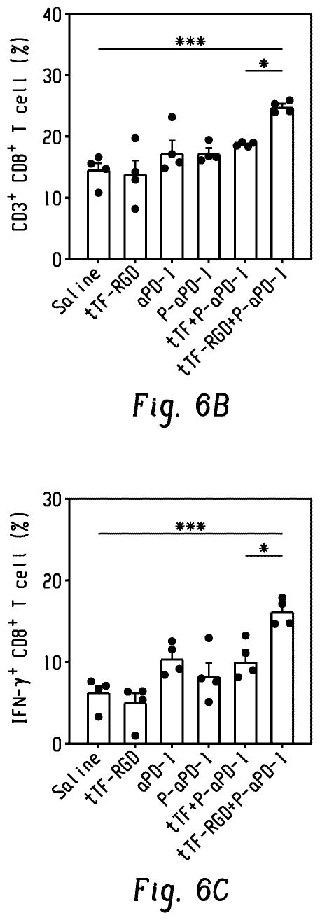

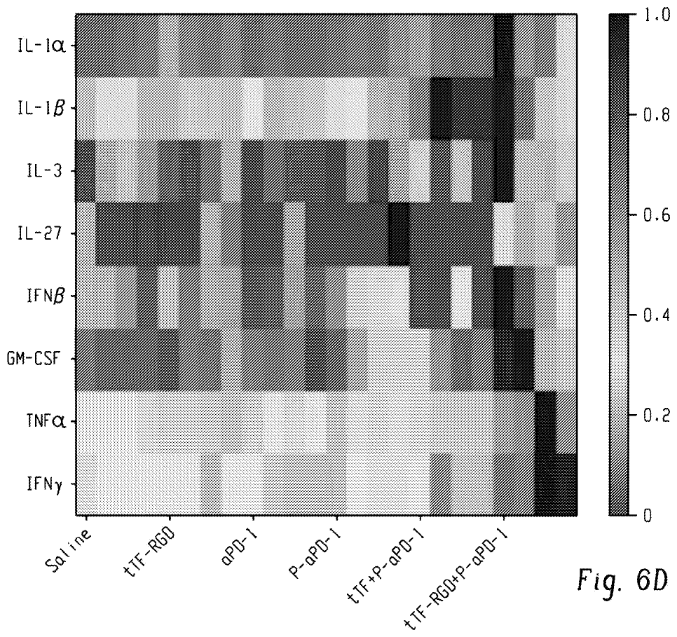

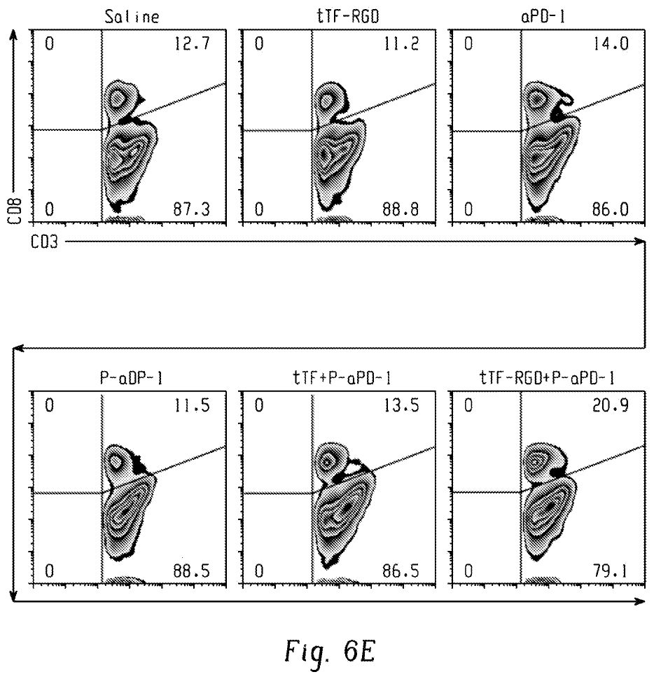

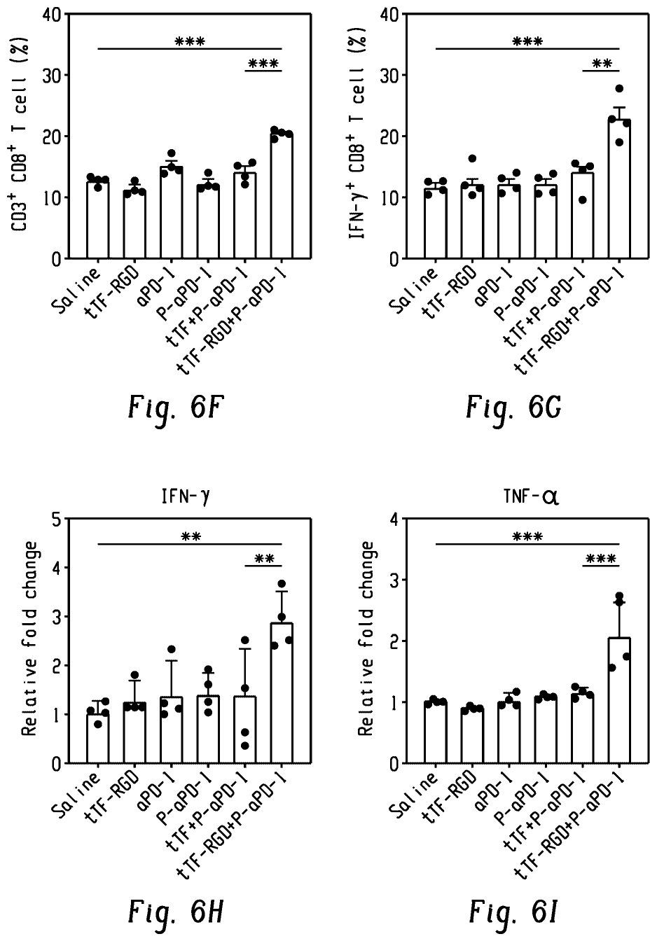

To investigate the underlying mechanism of the enhanced immunotherapy efficacy of the tTF-RGD+P-aPD-1 treatment strategy, the immune cell composition within tumor tissues was profiled with a focus on examining CD8⁺ effector T cell proportion. In the CT26 tumor-bearing mice after treatment with p.t. injection of tTF-RGD+i.v. injection of P-aPD-1, as shown in FIG. 6A, all the aPD-1-based treatments increased the percentages of CD8⁺ T cell populations in CD3⁺ T cells. Furthermore, tTF-RGD+P-aPD-1 treatment showed the best efficacy in increasing the proportion of CD3⁺CD8⁺ T cells among all treatment groups, which displayed a 1.33-fold increase compared to that in tTF+P-aPD-1 treatment group and a 1.69-fold increase compared to that in the saline group (FIG. 6B). Moreover, the proportions of IFN-γ+CD8⁺ T cells, which are recognized as effector CD8⁺T cells, were also remarkably increased after treatment of tTF-RGD+P-aPD-1, which displayed a 1.60-fold increase compared to that in tTF+P-aPD-1 treatment group and a 2.61-fold increase compared to that in the saline group (FIG. 6C). The cytokines in the tumor tissues were examined, and a marked elevation in anti-tumoral cytokines was found, such as TNF-α and IFN-γ, after tTF-RGD+P-aPD-1 treatment (FIG. 6D).

The changes in T cell populations after i.v. injection of tTF-RGD+i.v. injection of P-aPD-lin the CT26 tumor-bearing mice were also investigated. Due to the very low dose of aPD-1, treatment groups except for tTF-RGD+P-aPD-1 showed a negligible impact on CD8⁺CD3⁺ T cell populations (FIGS. 6E, F and G). On the contrary, tTF-RGD+P-aPD-1 remarkably increased CD3⁺CD8⁺ T cell populations, which displayed a 1.44-fold increase compared to that in tTF+P-aPD-1 treatment group and a 1.61-fold increase compared to that in the saline group (FIG. 6F). Moreover, the proportions of IFN-γ+CD8⁺T cells were remarkably increased after treatment of tTF-RGD+P-aPD-1, which displayed a 1.59-fold increase compared to that in tTF+P-aPD-1 treatment group and a 1.98-fold increase compared to that in the saline group (FIG. 6G). The levels of IFN-Y (FIG. 6H) and TNF-α (FIG. 6I) were examined by ELISA assay. The results demonstrated a 2.83-fold increase in IFN-γ levels and a 2.04-fold increase in TNF-α level in tTF-RGD+P-aPD-1 treatment group compared with saline group. The results suggested that tTF-RGD+P-aPD-1 treatment promoted the secretion of these anti-tumoral cytokines.

### Example 5: Recruitment of PM-NP to Tumor Site by tTF-RGD in a PDX Model

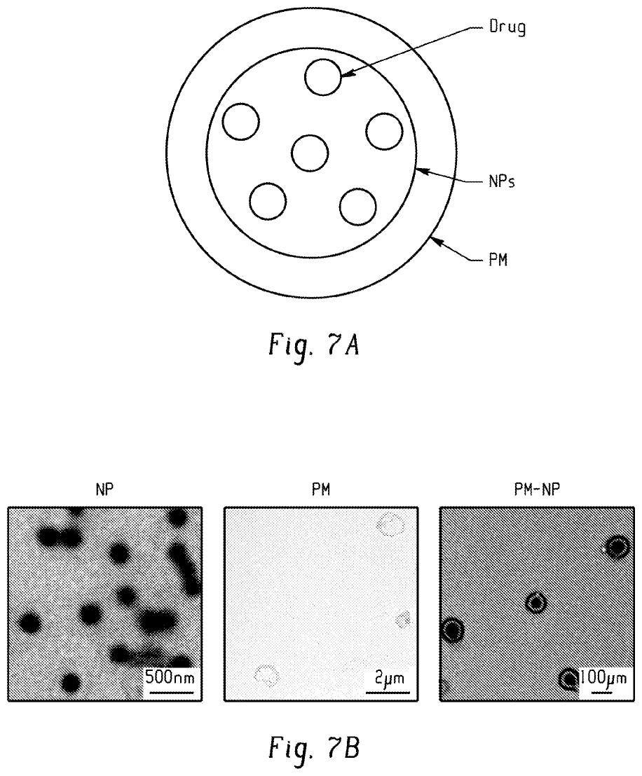

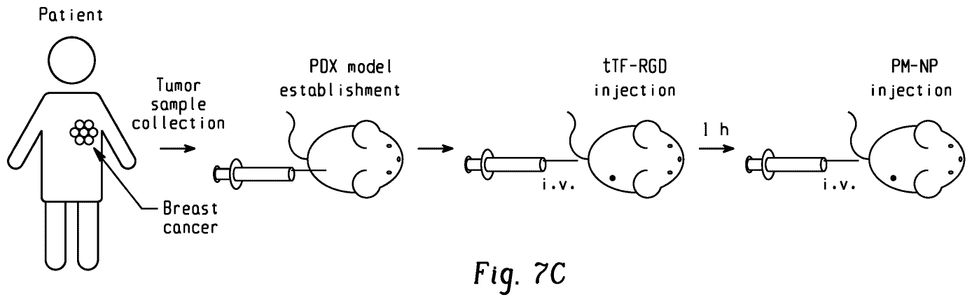

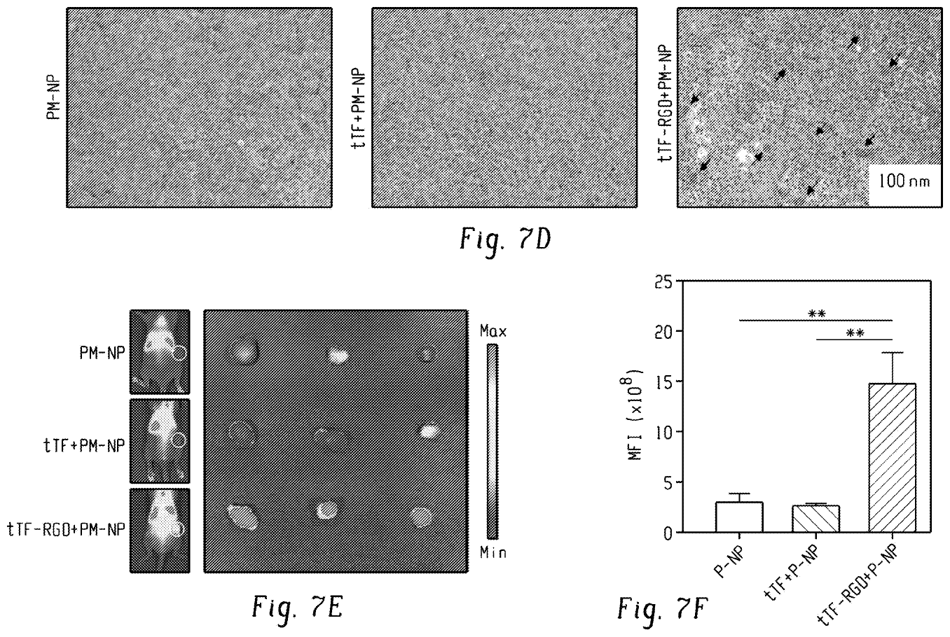

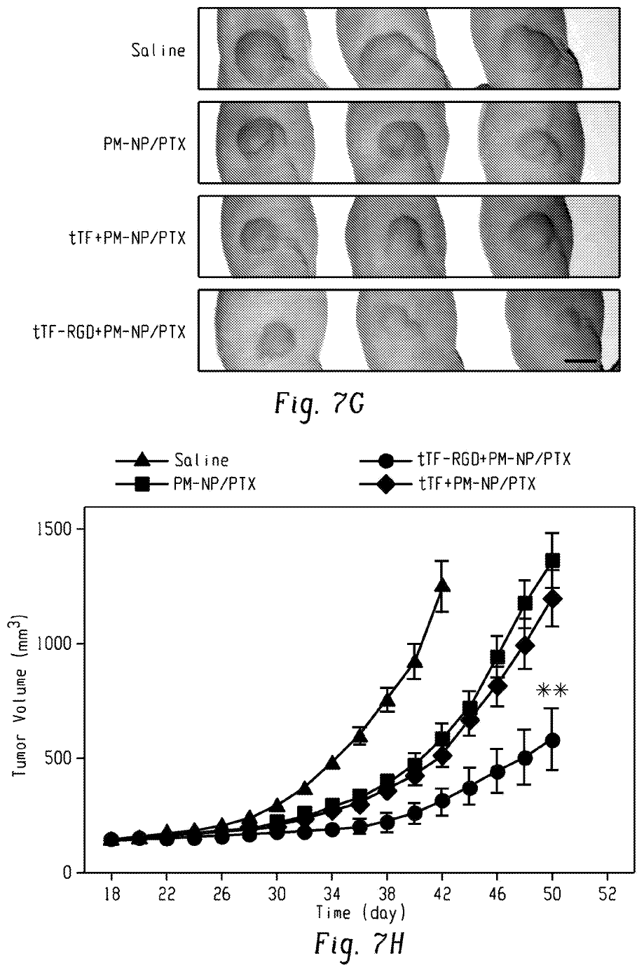

Platelet membranes inherit many surface receptors from platelets, and previous reports demonstrated that platelet membranes-cloaked nanoparticles could migrate in response to platelet-attracting signal. Therefore, it was hypothesized that the tTF-RGD-mediated platelet-recruiting strategy triggered by tumor-selective thrombosis could also be leveraged to guide the migration and accumulation of platelet membranes-cloaked nanoparticles. To test this, modified dextran-based NPs were prepared using a single-emulsion method, and then camouflaged with platelet membranes (designated as PM-NP) as reported in the art (FIG. 7A). The size of the NP and PM-NP were characterized by DLS, which showed average sizes of 170.1 nm and 178.8 nm in diameter, respectively (data not shown). There was a slight increase in the size of NP after the decoration of platelet membranes. TEM images showed a transparent layer outside the NPs, suggesting the successful coating of the platelet membrane onto NPs (FIG. 7B).

Next, it was determined if tTF-RGD can recruit PM-NP to tumor sites. To validate the translational potential of our treatment strategy, a clinically relevant PDX mouse model was established (FIG. 7C). TM00096 human breast tumor tissues were implanted on the flank of the immunodeficient mice. To affirm that systemic injection of tTF-RGD can initiate the tumor-selective coagulation on the PDX model, tTF+PM-NP or tTF-RGD+PM-NP were administered to the mice via i.v. injection as described above in the CT26-bearing mice model. Twenty-four hours later, the PDX tumors were collected and subjected to H&E staining. The results demonstrated that tTF-RGD could induce coagulation in PDX tumor tissues without severe hemorrhage (FIG. 7D). In order to visualize the tumor-selective accumulation of PM-NP, DiR dyes were encapsulated into NPs, which were further coated with platelet membranes. The mice bearing PDX tumors received i.v. injection of tTF or tTF-RGD. One hour later, DiR-loaded PM-NP was injected via i.v. route. From the IVIS images shown in FIG. 7E, mice treated with tTF-RGD+PM-NP showed markedly enhanced accumulation of PM-NP at the tumor site at 24 h compared to tTF+pM-NP and PM-NP treatment groups. Furthermore, the ex vivo quantification of MFI of tumors showed a 5.05-fold increase in the accumulation of PM-NP in mice treated with tTF-RGD than in those who received tTF treatment (FIG. 7F). Collectively, these results demonstrate that tTF-RGD that can induce tumor-specific coagulation was capable of enhancing the accumulation of PM-NPs at the tumor site.

### Example 6: In Vivo Anti-Tumor Efficacy Of tTF-RGD+PTX-Loaded PM-NP in a PDX Model

The encouraging results of the augmented accumulation of PM-NP after tTF-RGD treatment motivated the examination of its impact on the anti-tumor effect of the drug-loaded PM-NP. To expand the application of the treatment strategy to different treatment modalities, chemotherapeutic PTX-loaded PM-NP (designated PM-NP/PTX) were prepared and the therapeutic efficacy of tTF-RGD+PM-NP/PTX was tested in the PDX model. The TM00096 breast cancer PDX model was established on immunodeficient mice. When the tumor volumes reached to 100-150 mm³, the mice received different treatments, including saline, PM-NP/PTX, tTF+PM-NP/PTX, and tTF-RGD+PM-NP/PTX (tTF and tTF-RGD doses: 1.25 mg/kg; PTX dose: 2.5 mg/kg). As shown in FIG. 7G, 7H, PDX tumor-bearing mice reached the maximized tumor burden allowed in the animal protocol (1500 mm³) around day 40, while all the treatment groups with PM-NP/PTX suppressed tumor growth to various extents. Of note, tTF-RGD+PM-NP/PTX exhibited markedly better inhibition of tumor growth when compared to PM-NP/PTX and tTF+PM-NP/PTX. Notably, the average tumor sizes in tTF-RGD+PM-NP/PTX on day 50 were 587.72±135.29, which was significantly smaller than those in tTF+PM-NP/PTX (1202.27±125.58) and PM-NP/PTX (1365.03±120.06). No substantial weight loss in the mice was observed after tTF-RGD+PM-NP/PTX treatment, suggesting the acceptable safety profile of this treatment. Taken together, the tTF-RGD-mediated coagulation facilitates the tumor accumulation of PM-NP/PTX, contributing to superior therapeutic efficacy.

### Discussion of Examples

Described herein is the use of tTF-RGD to initiate the coagulation cascade at the tumor site, which can recruit P-aPD-1 and platelet membrane-coated nanoparticles. tTF-RGD targeted tumor neovasculature and initiated a coagulation cascade by mimicking the functionality of the tissue factors. The tTF-RGD injected either by peritumoral or intravenous injection showed potent capability in triggering thrombus formation to the tumor region. P-aPD-1 was thereby recruited to the tumor area and subsequently activated to secrete PMPs and release aPD-1 antibodies locally. The strategy remarkably suppressed CT26 tumor growth and prolonged the survival time of CT26 tumor-bearing mice. Flow cytometry analysis revealed that the treatment increased CD3⁺CD8⁺ T cell populations and the proportions of IFN-γ+CD8⁺ T cells. It was also explored if platelet membrane-coated nanoparticles can also migrate in response to tTF-RGD-mediated coagulation signaling. PTX-loaded PM-NP/PTX were prepared the tumor accumulation and antitumoral efficacy after tTF-RGD treatment were tested in a PDX breast cancer model. The results demonstrated that PM-NP/PTX can efficiently accumulate at the tumor site which was attributed to the attraction of platelets and platelet derivatives by thrombosis. Furthermore, this enhanced accumulation led to increased drug availability at the tumor site, thereby improving chemotherapy efficacy against PDX breast cancer.

In comparison with conventional targeting strategies based on ligand-receptor interaction, the tTF-RGD-mediated targeting strategy is expected to be less restricted by receptor saturation. By occupying a portion of receptors on cells, tTF-RGD can amplify the signals by inducing coagulation in the tumor area to attract substantial platelet-based therapeutics. Similar strategies may have the potential to apply to tumors with certain heterogeneity in expressing antigens or targeting receptors. Even if only a small portion of tumor cells express a receptor, coagulation can take place using the receptor-targeted tTF and thus the signals can be amplified in the tumor area. Both the dose and the administration intervals between tTF-RGD and platelets can be adjusted, which provides flexible and dynamic control over the migration of platelets-based therapeutics.

Cells are naturally smart components that can recognize and respond to physiological cues, which creates enthusiasm for developing cell-based delivery systems. Various surface receptors or other molecules enable cells to respond to physiological stimuli, allowing them to spontaneously target disease area such as inflammatory or tumor sites. However, these properties can also restrict the potential of the cells, as their actions depend on the naturally existing cues. As a result, it remains difficult to communicate with cells or precisely manipulate their migration. For example, previous studies on the platelet-based delivery system are focused on preventing post-surgery tumor recurrence because the formed chronic inflammatory environment secondary to the surgical resection is favorable for platelet targeting and activation. Described herein is a strategy to navigate the migration of platelets by inducing tumor-specific coagulation.

For clinical translation, many cell-based therapies such as CAR-T therapy are under clinical trial or have been approved for clinical use. Specifically, platelet infusion has been tested for decades and it can be anticipated that platelet-based therapy may also exhibit good biosafety. To test if tTF-RGD can enhance the treatment outcome, an FDA-approved chemodrug PTX was loaded into PM-NP and its therapeutic efficacy was tested in a PDX model. The results provide initial evidence that this strategy has potent efficacy and a good biosafety profile on a patients-related model. Furthermore, the cargoes in this versatile system can be replaced with other clinically available drugs, suggesting its promising wide application.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of treating a human patient with a solid tumor, comprising administering to the human patient an effective amount of a fusion polypeptide comprising a tissue factor fragment and a covalently linked tumor vessel endothelial cell binding peptide, wherein the effective amount activates coagulation at the solid tumor site;
waiting for a period of time; and
administering to the human patient an effective amount of a composition comprising a platelet cell or a platelet membrane,
wherein the platelet cell comprises a covalently linked immune checkpoint inhibitor, and
wherein the platelet membrane coats a chemotherapeutic agent nanoparticle.

2. The method of claim 1, wherein the tissue factor fragment comprises amino acids 1-210 to 1-218 of SEQ ID NO: 1 or a variant with at least 95% sequence identity to SEQ ID NO: 1 which functions to activate blood clotting, specifically tTF₁₋₂₁₈, (SEQ ID NO: 2).

3. The method of claim 1, wherein the tumor vessel endothelial cell binding peptide comprises GRGDSP (SEQ ID NO:3), GNGRAHA (SEQ ID NO:4), GALNGRSHAG (SEQ ID NO:5), GCNGRCG (SEQ ID NO:6), GCNGRCVSGCAGRC (SEQ ID NO: 7) or GCVLNGRMEC (SEQ ID NO:8).

4. The method of claim 1, wherein the tissue factor fragment and the tumor vessel endothelial cell binding peptide are covalently linked by a 1 to 15 amino acid linker.

5. The method of claim 1, wherein the immune checkpoint inhibitor comprises an antibody, antibody fragment or peptide that specifically binds CD25, PD-1, PD-L1, PD-L2, CTLA-4, immunoglobulin receptor (KIR), LAG-3, TIM-3, 4-1BB, 4-1BBL, GITR, CD40, CD40L, OX40, OX40L, CXCR2, B7-H3, B7-H4, BTLA, HVEM, CD28, A2aR, CD27, CD70, TCR ICOS, CD80, CD86, ICOS-L, CD70, Gal-9, VISTA, CD-137, CD155, CD266, PVR, PVR-2, CD47, CD160, NT5E, CD96, or TNFRSF18.

6. The method of claim 5, wherein the immune checkpoint inhibitor is an anti-PD-1 antibody.

7. The method of claim 1, wherein the immune checkpoint inhibitor is conjugated to the platelet cell via a bifunctional linker.

8. The method of claim 1, wherein the chemotherapeutic agent nanoparticle comprises a biodegradable nanoparticle, the biodegradable nanoparticle comprising poly-lactic acid (PLA); poly-D-L-glycolide (PLG); poly-D-L-lactide-co-glycolide (PLGA), poly-alkyl-cyanoacrylate (PCA), poly-ε-caprolactone, gelatin, alginate, chitosan, agarose, a polysaccharide, a protein, or a combination thereof.

9. The method of claim 8, wherein the biodegradable nanoparticle comprises a dextran.

10. The method of claim 1, wherein the chemotherapeutic agent comprises paclitaxel.

11. The method of claim 1, wherein the fusion polypeptide is administered by local administration, and the period of time is 30 minutes to 1 hour.

12. The method of claim 11, wherein local administration is peritumoral injection.

13. The method of claim 1, wherein the fusion polypeptide is administered by systemic administration, and the period of time is 60 minutes to 2 hours.

14. The method of claim 13, wherein systemic administration is intravenous injection.

15. The method of claim 1, wherein the composition comprising the platelet cell or the platelet membrane is administered by systemic administration.

16. The method of claim 15, wherein local administration is peritumoral injection and systemic administration is intravenous injection.

17. The method of claim 1, wherein the solid tumor is in bladder, breast, cervix, colon, rectal, endometrial, kidney, oral, liver, lung, melanoma, non-small cell lung cancer, ovarian, pancreatic, prostate, sarcoma, small cell lung cancer, or thyroid.

18. The method of claim 1, wherein the solid tumor is a metastatic tumor.
